(19) United States
(12) Patent Application Publication
(10) Pub. No.: US 2012/0220720 A1
(43) Pub. Date: Aug. 30, 2012

(54) FUNCTIONALIZED CELL BINDING PEPTIDES AND CELL CULTURE ARTICLES

(75) Inventors: Dana Craig Bookbinder, Corning, NY (US); Arthur Winston Martin, Horseheads, NY (US); Jodelle Karen Nelson, Corning, NY (US); Shawn Michael O'Malley, Horseheads, NY (US); Yue Zhou, Horseheads, NY (US)

(73) Assignee: Corning Incorporated

(21) Appl. No.: 13/121,825

(22) PCT Filed: Jul. 29, 2010
(86) PCT No.: PCT/US10/43626

§ 371 Date: Mar. 30, 2011

Related U.S. Application Data
(60) Provisional application No. 61/229,520, filed on Jul. 29, 2009.

(51) Int. Cl. C08F 8/00; C08G 65/48; C07K 14/00; C08G 59/14; C07K 7/08; C07K 7/06

(52) U.S. Cl. 525/54.1; 530/326; 530/327; 530/329; 530/330

(57) ABSTRACT

Synthetic surfaces capable of supporting culture of cells in culture, particularly cells that will be used therapeutically, are disclosed. The synthetic cell culture surfaces have a functionalized peptide, a peptide that has been functionalized to contain a polymerization moiety, and optionally a spacer, grafted to a hydrophilic polymeric base material. Methods of making the surfaces and methods of using the surfaces are also disclosed.

### CLAIMING BENEFIT OF PRIOR FILED U.S. APPLICATION

This application claims the benefit of U.S. Provisional Application Ser. No. 61/229,520, filed on Jul. 29, 2009. The content of this document and the entire disclosure of publications, patents, and patent documents mentioned herein are incorporated by reference.

### FIELD

The present disclosure relates to functionalized cell binding peptides and their use in preparing cell culture articles. More particularly, the disclosure relates to synthetic surfaces and articles for supporting the culture of undifferentiated stem cells in chemically defined medium.

### SEQUENCE LISTING

This application contains a Sequence Listing electronically submitted via EFS-Web to the United States Patent and Trademark Office as text filed named “20100723_SP09-223_Sequence_listing.txt” having a size of 8 kb and created on Jul. 23, 2010. Due to the electronic filing of the Sequence Listing, the electronically submitted Sequence Listing serves as both the paper copy required by 37 CFR §1.821(c) and the CRF required by §1.821(e). The information contained in the Sequence Listing is hereby incorporated herein by reference.

### BACKGROUND

Therapeutic cells, cells which may be introduced into a human for the treatment of disease, are being developed. Examples of therapeutic cells include pluripotent stem cells such as human embryonic stem cells (hESCs) which have the ability to differentiate into any of the three germ layers, giving rise to any adult cell type in human body. This property of stem cells provides a potential for developing new treatments for a number of serious cell degenerative diseases, such as diabetes, spinal chord injury, heart diseases and the like. However there remain obstacles in the development of such hESC-based treatments. Obtaining and maintaining adequate numbers of therapeutic cells in cell and tissue culture and ensuring that these cells do not change in unwanted ways during cell culture are important in developing and controlling therapeutic cell cultures. For example, stem cell cultures, such as hESC cell cultures are typically seeded with a small number of cells from a cell bank or stock and then amplified in the undifferentiated state until differentiation is desired for a given therapeutic application. To accomplish this, the hESC or their differentiated cells are currently cultured in the presence of surfaces or media containing animal-derived components, such as feeder layers, serum, or Matrigel™ available from BD Biosciences, Franklin Lakes N.J. These animal-derived additions to the culture environment expose the cells to potentially harmful viruses or other infectious agents which could be transferred to patients or compromise general culture and maintenance of the hESCs. In addition, such biological products are vulnerable to batch variation, immune response and limited shelf-life.

### SUMMARY

In embodiments, a functionalized peptide is provided comprising a cell adhesive peptide which contains a cell binding sequence, at least one polymerization moiety wherein the polymerization moiety is an α, β un-saturated group or ethylenically unsaturated group which is, for example, acrylate, methacrylate, acrylamide, methyacrylamide, maleimide, fumarates or epoxides, and a spacer moiety wherein the spacer moiety is a polyethylene oxide, Xaaₙ where Xaa is independently any amino acid and where n is an integer from 0 to 3, from 0 to 6, from 0 to 10, from 0 to 20 or from 0 to 30, or combinations, and wherein the polymerization moiety is bound to the cell adhesive peptide or the spacer moiety.

In embodiments, the cell adhesive peptide comprises the sequence:

| | |
|---|---|
| KGGGQKCIVQTTSWSQCSKS; | (SEQ ID NO: 1) |
| GGGQKCIVQTTSWSQCSKS; | (SEQ ID NO: 2) |
| KYGLALERKDHSG; | (SEQ ID NO: 3) |
| YGLALERKDHSG; | (SEQ ID NO: 4) |
| KGGSINNNRWHSIYITRFGNMGS; | (SEQ ID NO: 5) |
| GGSINNNRWHSIYITRFGNMGS; | (SEQ ID NO: 6) |
| KGGTWYKIAFQRNRK; | (SEQ ID NO: 7) |
| GGTWYKIAFQRNRK; | (SEQ ID NO: 8) |
| KGGTSIKIRGTYSER; | (SEQ ID NO: 9) |
| GGTSIKIRGTYSER; | (SEQ ID NO: 10) |
| KYGTDIRVTLNRLNTF; | (SEQ ID NO: 11) |
| YGTDIRVTLNRLNTF; | (SEQ ID NO: 12) |
| KYGSETTVKYIFRLHE; | (SEQ ID NO: 13) |
| YGSETTVKYIFRLHE; | (SEQ ID NO: 14) |
| KYGKAFDITYVRLKF; | (SEQ ID NO: 15) |
| YGKAFDITYVRLKF; | (SEQ ID NO: 16) |
| KYGAASIKVAVSADR; | (SEQ ID NO: 17) |
| YGAASIKVAVSADR; | (SEQ ID NO: 18) |
| CGGNGEPRGDTYRAY; | (SEQ ID NO: 19) |
| GNGEPRGDTYRAY; | (SEQ ID NO: 20) |
| CGGNGEPRGDTRAY; | (SEQ ID NO: 21) |
| GGNGEPRGDTRAY; | (SEQ ID NO: 22) |
| KYGRKRLQVQLSIRT; | (SEQ ID NO: 23) |
| YGRKRLQVQLSIRT; | (SEQ ID NO: 24) |
| KGGRNIAEIIKDI; | (SEQ ID NO: 25) |
| GGRNIAEIIKDI; | (SEQ ID NO: 26) |
| KGGPQVTRGDVFTMP; | (SEQ ID NO: 27) |
| GGPQVTRGDVFTMP; | (SEQ ID NO: 28) |
| GRGDSPK; | (SEQ ID NO: 29) |
| KGGAVTGRGDSPASS; | (SEQ ID NO: 30) |
| GGAVTGRGDSPASS, | (SEQ ID NO: 31) |
| Yaa₁PQVTRGNVFTMP | (SEQ ID NO: 32) |
| or | |
| RGDYK. | (SEQ ID NO: 33) |

In embodiments, a cell culture article is provided comprising a functionalized peptide covalently linked to a hydrophilic polymeric base material, wherein the functionalized peptide is described by the formula: Rₘ—Sₚ—Cₐₚ. R is a polymerization moiety, an α, β unsaturated group or ethylenically unsaturated group which includes acrylate, methacrylate, acrylamide, methyacrylamide, maleimide, fumarate, or epoxide, which is capable of polymerizing in the presence of an external energy source such as UV or visible light with an optional catalyst, or by thermal polymerization with an optional catalyst. “m” is an integer greater than or equal to 1. Sₚ is a spacer. In embodiments, Sₚ may be a polyethylene oxide having the formula (O—CH₂CHR′)ₘ₂ where R′ is H or CH₃ and m2 is an integer from 1 to 20. For example, Sp may be polyethylene glycol (PEG) or polypropylene glycol (PPG). In embodiments, the polyethylene oxide spacer may be of any length. For example Sₚ may be PEG₂, PEG₄, PEG₆, PEG₈, PEG₁₀, PEG₁₂ or PPG₂, PPG₄, PPG₆, PPG₈, PPG₁₀, PPG₁₂ or PPG₂₀. In embodiments, Sₚ may be an amino acid Xaaₙ where Xaa is any amino acid and n is an integer from 0 to 30, from 0 to 20, from 0 to 10, 0 to 6, or from 0 to 3, or combinations of Xaa and polyethylene oxide. Xaaₙ may comprise a lysine, glysine, glutamic acid, serine, aspartic acid or arginine amino acid, which may be a terminal amino acid. In embodiments, Xaaₙ is a hydrophilic amino acid. In embodiments, Xaa is lysine and n is greater than 1. Or, in embodiments, the spacer Sₚ may comprise polyethylene oxide spacer and amino acid spacer in any combination. The polymerization moiety may attach to the spacer, Sₚ through the polyethylene oxide, through the side chain of an amino acid such as lysine or at the N-terminus of the amino acid. Amino acid Xaaₙ may be acetylated and/or amidated to protect it from degradation. However, if Xaaₙ is acetylated, the polymerization moiety cannot be bound to Xaaₙ through the N-terminus of the amino acid. Cₐₚ is a peptide or polypeptide which has a cell binding or cell adhesive sequence.

The hydrophilic polymeric base material comprises hydrophilic monomers. In embodiments the hydrophilic monomers are, for example, N-Tris(hydroxymethyl)acrylamide (ACRYLNTRIS-OH) and copolymer, glyceryl monomethacrylate (GLY-METH), poly(serine)methacrylate and copolymer (SER-METH). In these embodiments copolymers are formed by cross-linking with the following di-functional moieties which are acrylamides and/or acrylates: N,N′-(1,2-dihydoxyethylene)bisacrylamide and glycerol 1,3-diglycerolate diacrylate. In embodiments, the cell culture article has a contact angle of less than 50°. In further embodiments, the spacer Sₚ is PEO₄.

In additional embodiments, a method is provided for making a cell culture surface comprising the steps of: providing a hydrophilic base material comprising hydrophilic monomers to a substrate surface; polymerizing the hydrophilic base material; providing a functionalized peptide to the surface of polymerized or cured hydrophilic base material; polymerizing the functionalized peptide to the hydrophilic base material; and, optionally washing. In embodiments of the method, the hydrophilic base material comprises N-Tris(hydroxymethyl)acrylamide (ACRYLNTRIS-OH), glyceryl monomethacrylate (GLY-METH), poly(serine)methacrylate (SER-METH), hydroxyethylmethylacrylate (HEMA), or acrylamide (ACRYL) polymers and copolymers and optionally hydrophilic crosslinking materials such as, N,N′-(1,2-dihydroxyethylene)bisacrylamide, glycerol 1,3-diglycerolate diacrylate, or combinations thereof.

In embodiments of the method, the functionalized peptide is described by the formula: Rₘ—Sₚ—Cₐₚ. Rₘ is a polymerization moiety, an α, β unsaturated group or ethylenically unsaturated group which includes acrylate, methacrylate, acrylamide, methyacrylamide, maleimide, fumarate, or an epoxide. Rm is capable of polymerizing for example, in the presence of an external energy source such as UV or visible light with an optional catalyst, or by thermal polymerization with an optional catalyst. “m” is an integer greater than or equal to 1. Sₚ is a spacer. In embodiments, Sₚ may be a polyethylene oxide including for example polyethylene glycol (PEG) or polypropylene glycol (PPG). For example Sₚ may be PEG₄. In embodiments, Sₚ may be an amino acid Xaaₙ where Xaa is any amino acid and n is an integer from 0 to 30, from 0 to 20, from 0 to 10 or from 0 to 3, or combinations of Xaa and polyethylene oxide. Xaaₙ may comprise a lysine or arginine amino acid, which may be a terminal lysine or arginine. Or, in embodiments, the spacer Sₚ may comprise polyethylene oxide spacer and amino acid spacer in any combination. The polymerization moiety may attach to the spacer, Sₚ through the polyethylene oxide, through the side chain of an amino acid such as lysine or at the N-terminus of the amino acid. Or, the polymerization moiety may attach to the cell adhesive peptide Cₐₚ through the side chain of an amino acid such as lysine or at the N-terminus of the amino acid. Amino acid Xaaₙ or Cₐₚ may be acetylated and/or amidated to protect it from degradation. However, if Xaaₙ is acetylated, the polymerization moiety cannot be bound to Xaaₙ through the N-terminus of the amino acid. Cₐₚ is a peptide or polypeptide which has a cell binding or cell adhesive sequence.

In embodiments of the method, the cell adhesive peptide is:

| | |
|---|---|
| KGGGQKCIVQTTSWSQCSKS; | (SEQ ID NO: 1) |
| GGGQKCIVQTTSWSQCSKS; | (SEQ ID NO: 2) |
| KYGLALERKDHSG; | (SEQ ID NO: 3) |
| YGLALERKDHSG; | (SEQ ID NO: 4) |
| KGGSINNNRWHSIYITRFGNMGS; | (SEQ ID NO: 5) |
| GGSINNNRWHSIYITRFGNMGS; | (SEQ ID NO: 6) |
| KGGTWYKIAFQRNRK; | (SEQ ID NO: 7) |
| GGTWYKIAFQRNRK; | (SEQ ID NO: 8) |
| KGGTSIKIRGTYSER; | (SEQ ID NO: 9) |
| GGTSIKIRGTYSER; | (SEQ ID NO: 10) |
| KYGTDIRVTLNRLNTF; | (SEQ ID NO: 11) |
| YGTDIRVTLNRLNTF; | (SEQ ID NO: 12) |
| KYGSETTVKYIFRLHE; | (SEQ ID NO: 13) |
| YGSETTVKYIFRLHE; | (SEQ ID NO: 14) |
| KYGKAFDITYVRLKF; | (SEQ ID NO: 15) |
| YGKAFDITYVRLKF; | (SEQ ID NO: 16) |
| KYGAASIKVAVSADR; | (SEQ ID NO: 17) |
| YGAASIKVAVSADR; | (SEQ ID NO: 18) |
| CGGNGEPRGDTYRAY; | (SEQ ID NO: 19) |
| GNGEPRGDTYRAY; | (SEQ ID NO: 20) |
| CGGNGEPRGDTRAY; | (SEQ ID NO: 21) |
| GGNGEPRGDTRAY; | (SEQ ID NO: 22) |
| KYGRKRLQVQLSIRT; | (SEQ ID NO: 23) |
| YGRKRLQVQLSIRT; | (SEQ ID NO: 24) |
| KGGRNIAEIIKDI; | (SEQ ID NO: 25) |
| GGRNIAEIIKDI; | (SEQ ID NO: 26) |
| KGGPQVTRGDVFTMP; | (SEQ ID NO: 27) |
| GGPQVTRGDVFTMP; | (SEQ ID NO: 28) |
| GRGDSPK; | (SEQ ID NO: 29) |
| KGGAVTGRGDSPASS; | (SEQ ID NO: 30) |
| GGAVTGRGDSPASS; | (SEQ ID NO: 31) |
| Yaa₁PQVTRGNVFTMP; | (SEQ ID NO: 32) |
| RGDYK | (SEQ ID NO: 33) |
| or combinations. | |

In embodiments of the present invention, a method is described for culturing an isolated population of undifferentiated human embryonic stem cells in chemically defined medium on a synthetic culture surface.

### BRIEF DESCRIPTION OF THE DRAWINGS

FIG. 1 is a flow chart showing an embodiment of a method of making cell culture surfaces.

FIG. 2 is an illustration showing a method for making an embodiment of a cell culture surface.

FIG. 3 is another illustration showing a method for making an embodiment of a cell culture surface.

FIG. 4 is a bar graph showing contact angles measured from the base materials.

FIG. 5 shows the fluorescence intensity of fluorescently labeled functionalized peptide (having PEO spacer)—grafted to base materials.

FIG. 6A-F illustrates fluorescence measurements taken from Rhodamine-labeled functionalized peptide (MAA-PEG₄-SEQ ID NO:27-NH₂) on a GLY-METH base material (FIGS. 6A-C) and on a HEMA surface (FIGS. 6D-F).

FIG. 7A-F show photomicrographs of H7 crystal violet-stained human embryonic stem cells cultured on control surfaces Matrigel™ and Synthemax™, and on HEMA and Glycerol Methacrylate functionalized peptide-grafted surfaces, VN-MAA grafted to HEMA in FIG. 7C, VN-MAA grafted to glycerol in FIG. 7D, VN-PEG4-MAA grafted to HEMA in FIG. 7E and VN-PEG4-MAA grafted to Glycerol in FIG. 7F in embodiments of the present invention.

FIG. 8A-C are photomicrographs of H7 human embryonic stem cells cultured on control surfaces MG (Matrigel™, FIG. 8A and Synthemax™, FIG. 8B) and on the glycerol VN-PEO4-MAA surface (FIG. 8C) surface in embodiments of the present invention.

FIG. 9 shows XPS data showing binding energy of detected oxygen in HEMA surfaces.

FIG. 10 shows XPS data showing binding energy of detected oxygen in GLY-METH surfaces.

### DETAILED DESCRIPTION

In embodiments, the disclosure provides a functionalized peptide having a polymerization moiety (Rₘ), a spacer moiety (Sₚ) and a cell adhesive peptide moiety (Cₐₚ) and its use in forming a cell culture article suitable for supporting cells in culture. In embodiments, the cell culture article formed from the functionalized peptide is suitable for supporting cells in culture in the absence of serum. In embodiments, the functionalized peptide has formula Rₘ—Sₚ—Cₐₚ wherein R is a polymerization moiety selected from the group consisting of acrylate, methacrylate, acrylamide, methyacrylamide, maleimide fumarate and epoxide and combinations, and m is an integer greater than 1; and, wherein Sₚ is a spacer moiety wherein the spacer moiety comprises polyethylene oxide or polypropylene oxide having the formula O—CH₂CHR′)ₘ₂ where R′ is H or CH₃ and m2 is an integer from 1 to 20, or Xaaₙ wherein Xaa is any amino acid and n is an integer from 0 to 3, from 0 to 6, from 0 to 10, from 0 to 20 or from 0 to 30, or a combination; and wherein Cₐₚ is a peptide comprising a cell adhesive sequence.

In the field of cell culture, culturing cells in a scalable fashion requires surfaces that are free of pathogens, relatively inexpensive, stable and reliable, and support long term culture of cells in culture. This is particularly true for cell culture aimed at providing therapeutic cells. That is, cell culture aimed at providing cells which will be introduced or re-introduced into a human for the treatment of disease. While current technology for cell culture includes surfaces that are derived from animal products such as Matrigel™, derived from mouse tumor extract, these surfaces are not desirable for support of cells that will be used therapeutically.

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

Synthetic cell culture surfaces including surfaces that incorporate synthetic or recombinant proteins or peptides, for example, are suitable for supporting cells that may be introduced into a human for the treatment of disease. Synthetic peptides and proteins often include cell adhesive sequences such as RGD. Polypeptide sequences are referred to herein by their one letter amino acid codes and by their three letter amino acid codes. These codes may be used interchangeably. Synthetic surfaces that reduce the amount of peptide required to support viable cells in culture are desirable, because peptides can expensive, and so surfaces requiring less peptide are less expensive. In addition, synthetic surfaces that are easy to manufacture, stable in storage, stable through sterilization procedures, and stable through long exposure to aqueous cell culture conditions are also desirable.

In embodiments of the present invention, peptides or polypeptides which have been modified or functionalized to carry a polymerization moiety such as an acrylate, methacrylate, acrylamide, methacrylamide, maleimide fumarate or epoxide are provided. For the purposes of this disclosure “functionalized peptide” means peptides which have been modified to incorporate polymerization moieties such as acrylate, methacrylate, acrylamide, methacryalmide, maleimide fumarate or epoxide groups. In embodiments these polymerization moieties can form polymers in the presence of an external energy source such as UV or visible light with an optional catalyst, or by thermal polymerization with an optional catalyst. In embodiments, the functionalized peptides or polypeptides may contain a spacer moiety.

In embodiments, the functionalized peptide is described by formula 1:

Rₘ—Sₚ—Cₐₚ Formula 1

In embodiments, Rₘ is a polymerization moiety, an α, β unsaturated group or ethylenically unsaturated group which includes acrylate, methacrylate, acrylamide, methyacrylamide, maleimide, fumarate, or an epoxide, which is capable of polymerizing in the presence of an external energy source. “m” is an integer greater than or equal to 1. Sₚ is a spacer. In embodiments, Sₚ may be a polyalkylene oxide including for example polyethylene glycol (PEG) or polypropylene glycol (PPG) which are represented by the formula O—CH₂CHR′)ₘ₂ where R′ is H or CH₃ and m2 is an integer from 1 to 20. In embodiments, relatively short chains of polyalkylene oxide are desirable. For example, in embodiments, Sₚ may be PEG₂, PEG₄, PEG₆, PEG₈, PEG₁₀, PEG₁₂ or PPG₂, PPG₄, PPG₆, PPG₈, PPG₁₀, PPG₁₂ or PPG₂₀.

Others have disclosed the use of (meth)acrylic acid derivatives chemically modified with a protein or peptide for the preparation of cell culture surfaces (U.S. Pat. No. 5,643,561, the '561 patent). In the '561 patent, no disclosure is made of the use of a spacer between the peptide sequence and the polymerization moiety. Others have disclosed the use of a long chain PEG spacer, combined with a cell adhesive peptide sequence and a polymerization moiety. For example Hem, D. L., and Hubbell, J. A., Incorporation of Adhesion Peptides into Nonadhesive Hydrogels Useful for Tissue Resurfacing, Journal of Biomedical Materials Research Part A Vol. 39, Issue 2, pp. 266-276 (Hern & Hubbell) discloses the use of cell adhesive peptides conjugated to a polymerization moiety, and the use of cell adhesive peptides conjugated to polymerization moiety via a long chain polyalkylene oxide spacer (PEG75) which was combined with PEG diacrylate (copolymerized with PEG diacrylate) to form a hydrogel cell culture surface composed primarily of PEG. However, Hem and Hubbell disclose that the use of a cell adhesive peptide conjugated directly to a polymerization moiety produced a cell culture surface that did not support the specific binding of cells to the cell binding protein sequences provided. That is, cells seeded on hydrogels containing peptide incorporated via a linkage lacking a PEG spacer arm adhered nonspecifically, i.e. in a manner that required serum proteins and did not depend on the precise identity of the peptide provided. This was not desirable, according to Hern&Hubble. The use of a long chain PEG spacer (MW3400 PEG), in combination with a predominantly PEG hydrogel polymeric material supported specific cell binding. Hem & Hubbell is silent as to the use of short chain PEG spacers, such as O—CH₂CHR)ₘ₂ where R′ is H or CH₃ and m2 is an integer from 1 to 20. In addition, the Hern & Hubbell disclosure relates to surfaces that are primarily composed of PEG. (see also U.S. Pat. No. 7,615,593 which discloses the use of bifunctional PEG of the formula ((CH2)m-O)n where m is an integer from 2 to 8 and n is an integer greater than 100, and preferably 2,000 (column 5, line 64 to column 6, line 2).

The surfaces disclosed herein have, in embodiments, glycerol methacrylate and/or HEMA hydrophilic base matrices to which functionalized peptides are bound. HEMA and glycerol methacrylate have different cell culture characteristics compared to PEG. PEG is a non-binding surface. That is, proteins do not adsorb to PEG, and cells to not bind to PEG surfaces. PEG, in general, has a contact angle of less than 20 degrees. HEMA and glycerol methacrylate are not as non-binding as PEG, and the contact angles of surfaces prepared according to embodiments disclosed herein have contact angles of between about 20 degrees and about 60 degrees. While not wishing to be bound by theory, it may be that these differences in overall composition allow the functionalized peptides disclosed herein to provide functional cell culture surfaces with specific cell binding characteristics, where the primarily PEG surfaces having peptides without a PEG spacer did not provide suitable cell culture surfaces as disclosed in Hem & Hubbell.

In embodiments Sp is PPG or PEG having a functional group. For example, the PEG or PPG spacer may have a maleimide, thiol, amine, silane, aldehyde, epoxide, isocyanate, acrylate or carboxyl group. In embodiments the PEG spacer is a Jeffamine, a PEG having an amine functional group. In additional embodiments, the PEG or PPG may be branched. For example the branched PEG or PPO may be a Y-branched or star-PEG or PPG. In embodiments these branched PEG or PPO spacers may allow multiple peptides to be conjugated to a base material through a single functional peptide.

In embodiments, Sₚ may be an amino acid Xaaₙ where Xaa is independently any amino acid and n is an integer from 0 to 30, from 0 to 20, from 0 to 10, from 0 to 6 or from 0 to 3. For example, in embodiments, Sₚ may be an amino acid Xaaₙ where Xaa is G and where n=1 to 20, or Sₚ may be an amino acid Xaaₙ where Xaa is K and n=1 to 20 or Xaa is K and n=n is greater than or equal to 1, or Sₚ may be an amino acid Xaaₙ where Xaa is D and n=1 to 20, or Sₚ may be an amino acid Xaaₙ where Xaa is E and n=1 to 20. For example, the functionalized peptide may be MAA-Lys-Lys-Lys-Lys-Lys-Lys-Lys-VN-Peptide (n-terminal attachment to lysine alpha terminal) or Ac-(Lys-Lys-Lys-Lys-Lys-Lys-MAA)-VN-Peptide (Methacrylate linked) to a series of lysine spacer length sprung from a epsilon lysine side chain. The MAA can be attached on the n-terminal of the spacer length or it can be formed on a lysine side chain. In embodiment, spacer Sₚ may be a three amino acid sequence such as LysGlyGly or LysTyrGly. In embodiments, Xaaₙ is a series of the same amino acid. In embodiments, the spacer Sₚ may be combinations of Xaaₙ and polyethylene or polypropylene oxide. Xaaₙ may comprise a hydrophilic amino acid such as lysine, glycine, glutamic acid, aspartic acid or arginine amino acid. In embodiments, Xaaₙ may have a terminal lysine or arginine. Or, in embodiments, the spacer Sₚ may comprise polyethylene oxide spacer and amino acid spacer in any combination. In embodiments, Sₚ may be a hydrophobic spacer such as palmitic acid, stearic acid, lauric acid or hexaethylene diamine (functionalized to allow the hydrophobic moiety to link both to the polymerization moiety and the peptide). In embodiments, Sₚ may be carboxyethyl methacrylate.

The polymerization moiety may attach to the spacer, Sₚ through the polyethylene oxide, through the side chain of an amino acid such as lysine or at the N-terminus of the amino acid. Amino acid Xaaₙ may be acetylated and/or amidated to protect it from degradation. However, if Xaaₙ is acetylated, the polymerization moiety cannot be bound to Xaaₙ through the N-terminus of the amino acid. Cₐₚ is a peptide or polypeptide which has a cell binding or cell adhesive sequence.

In embodiments, the spacer Sₚ is Xaaₙ and Xaaₙ has a terminal lysine. In embodiments, Xaaₙ may be bound to a polymerization moiety Rₘ. For example, Xaaₙ may be (MAA)LysGlyGly or (MAA)LysTyrGly, where MAA is the polymerization moiety methacrylic acid (MAA) bound to Xaaₙ through the side chain of the terminal lysine amino acid. In additional embodiments, the polymerization moiety may be bound to the N-terminus of the Xaaₙ amino acid or amino acid chain, if the N-terminus is not acetylated. Each functionalized peptide has at least one polymerization moiety, and may have more than one. Cₐₚ is a peptide or polypeptide having a cell adhesive or cell binding sequence:

In embodiments, the cell adhesive peptide or cell adhesive polypeptide (which terms are interchangeable) (Cₐₚ) has a cell binding or cell adhesive sequence which may, for example, be an integrin binding sequence or an RGD sequence. For the purposes of this disclosure, peptide or polypeptide is an amino acid sequence that may be chemically synthesized or made by recombinant methods. However, for the purposes of this disclosure, peptide or polypeptide is a fragment of a protein, and not a complete protein. In addition, peptide or polypeptide is not isolated from an animal source. In embodiments, peptide or polypeptide may include an amino acid sequence of Yaa₁ProGlnValThrArgGlyAspValPheThrMetPro (SEQ ID NO:32), a vitronectin peptide sequence where 1 is an integer from 0 to 3 and where Yaa may be any amino acid or may include, for example, lysine of which the terminal amino acid must be lysine or arginine to accommodate attachment of a polymerizable group. In embodiments, the peptide or polypeptide may be cyclic. For example RGDYK(SEQ ID NO:33) may be cyclic c(RGDyK).

Examples of peptides that may be used in embodiments are listed in Table 1.

TABLE 1

| Sequence | Source |
|---|---|
| KGGGQKCIVQTTSWSQCSKS | Cyr61 res 224-240 |
| (SEQ ID NO: 1) | |
| GGGQKCIVQTTSWSQCSKS | Cyr61 res 224-240 |
| (SEQ ID NO: 2) | |
| KYGLALERKDHSG | TSP1 res 87-96 |
| (SEQ ID NO: 3) | |
| YGLALERKDHSG | TSP1 res 87-96 |
| (SEQ ID NO: 4) | |
| KGGSINNNRWHSIYITRFGNMGS | mLMα1 res 2179-2198 |
| (SEQ ID NO: 5) | |
| GGSINNNRWHSIYITRFGNMGS | mLMα1 res 2179-2198 |
| (SEQ ID NO: 6) | |
| KGGTWYKIAFQRNRK | mLMα1 res 2370-2381 |
| (SEQ ID NO: 7) | |
| GGTWYKIAFQRNRK | mLMα1 res 2370-2381 |
| (SEQ ID NO: 8) | |
| KGGTSIKIRGTYSER | mLMγ1 res 650-261 |
| (SEQ ID NO: 9) | |
| GGTSIKIRGTYSER | mLMγ1 res 650-261 |
| (SEQ ID NO: 10) | |
| KYGTDIRVTLNRLNTF | mLMγ1 res 245-257 |
| (SEQ ID NO: 11) | |
| YGTDIRVTLNRLNTF | mLMγ1 res 245-257 |
| (SEQ ID NO: 12) | |
| KYGSETTVKYIFRLHE | mLMγ1 res 615-627 |
| (SEQ ID NO: 13) | |
| YGSETTVKYIFRLHE | mLMγ1 res 615-627 |
| (SEQ ID NO: 14) | |
| KYGKAFDITYVRLKF | mLMγ1 res 139-150 |
| (SEQ ID NO: 15) | |
| YGKAFDITYVRLKF | mLMγ1 res 139-150 |
| (SEQ ID NO: 16) | |
| KYGAASIKVAVSADR | mLMα1 res2122-2132 |
| (SEQ ID NO: 17) | |
| YGAASIKVAVSADR | mLMα1 res2122-2132 |
| (SEQ ID NO: 18) | |
| CGGNGEPRGDTYRAY | BSP |
| (SEQ ID NO: 19) | |
| GGNGEPRGDTYRAY | BSP |
| (SEQ ID NO: 20) | |
| CGGNGEPRGDTRAY | BSP-Y |
| (SEQ ID NO: 21) | |
| GGNGEPRGDTRAY | BSP-Y |
| (SEQ ID NO: 22) | |
| KYGRKRLQVQLSIRT | mLMα1 res 2719-2730 |
| (SEQ ID NO: 23) | |
| YGRKRLQVQLSIRT | mLMα1 res 2719-2730 |
| (SEQ ID NO: 24) | |
| KGGRNIAEIIKDI | LMβ1 |
| (SEQ ID NO: 25) | |
| GGRNIAEIIKDI | LMβ1 |
| (SEQ ID NO: 26) | |
| KGGPQVTRGDVFTMP | VN |
| (SEQ ID NO: 27) | |
| GGPQVTRGDVFTMP | VN |
| (SEQ ID NO: 28) | |
| GRGDSPK | Short FN |
| (SEQ ID NO: 29) | |
| KGGAVTGRGDSPASS | Long FN |
| (SEQ ID NO: 30) | |
| GGAVTGRGDSPASS | Long FN |
| (SEQ ID NO: 31) | |
| Yaa₁PQVTRGNVFTMP | VN |
| (SEQ ID NO: 32) | |
| RGDYK | RGD |
| (SEQ ID NO: 33) | |

In embodiments, the functionalized peptide has a polymerization moiety which may be a photopolymerizable moiety or a thermal polymerizable moiety. This polymerizable moiety may be, for example, an acrylate, methacrylate, acrylamide, methyacrylamide, maleimide fumarate or epoxide moiety. The polymerizable moiety may be bound to the Xaaₙ amino acid sequence through a side chain of the amino acid. For example, a methacrylic acid may be bound to a lysine amino acid through the side chain of the lysine amino acid where Sₚ is Xaaₙ, Xaa is lysine, n=1, Rₘ is methacrylic acid, and Cap is a peptide sequence, for example a peptide sequence shown in Table 1.

In embodiments, functionalized peptides have a spacer moiety Sₚ between the cell adhesive peptide (Cₐₚ) and the polymerization moiety Rₘ. The spacer may be a hydrophilic spacer, for example, polyethelene oxide (PEO), polyethylene glycol (PEG) or polypropylene oxide (PPO). The terms PEO and PEG can be used interchangeably. In embodiments, the spacer is PEO₄. The spacer may act to extend the peptide away from the cell culture surface, making the peptide more accessible to cells in culture, and improving the efficiency of the surface for cell culture. In addition, these hydrophilic spacers may act to repel proteins preventing non-specific absorption to the functionalized peptide. In embodiments, the use of a cell adhesive peptide with a spacer such as PEO (polyethylene oxide) in preparing cell culture articles allows for the preparation of such articles using a lower overall concentration of adhesive peptide. These functionalized peptides may be attached, covalently or non-covalently, to the base material.

Functionalized peptide or polypeptide may be conjugated to the base material at any density, preferably at a density suitable to support culture of cells. Functionalized peptide may be conjugated to base material at a density of between about 1 pmol per mm² and about 50 pmol per mm² of surface, which can be estimated by the surface area of base matrix that is coated in embodiments. For example, the functionalized peptide may be present at a density of greater 0.25 pmol/mm², greater than than 0.5pmol/mm², greater than 1 pmol/mm², greater than 5 pmol/mm², greater than 6 pmol/mm², greater than 7 pmol/mm², greater than 8 pmol/mm², greater than 9 pmol/mm², greater than 10 pmol/mm², greater than 12 pmol/mm², greater than 15 pmol/mm², or greater than 20 pmol/mm² of the base material surface. It will be understood that the amount of peptide present can vary depending on the composition of the base material, the thickness of the base material layer and the nature of the polypeptide itself. As discussed below in the Examples, higher densities of peptide may be better able to support attachment and proliferation of undifferentiated stem cells in a chemically defined medium, although other cell types may proliferate more successfully at different peptide densities.

In embodiments of the present invention, a base material is provided. For the purposes of this disclosure, “base material”, “hydrophilic base material” or “base material layer” (which terms are interchangeable) means a polymeric material to which a functionalized peptide is attached. In embodiments, the base material is a polymerized or semi-polymerized layer of monomers which provides moieties to allow for the attachment of functionalized peptides. In embodiments, the base material may be a hydrophilic base material. In embodiments, the hydrophilic base polymeric material may have a low contact angle. That is, the hydrophilic base polymeric layer may have a water contact angle of less than 60°, less than 55°, less than 50°, less than 40°, less than 30°, less than 20° or less than 10°. In embodiments, the hydrophilic base polymeric layer has a water contact angle less than 50°.

In embodiments, the base material is, for example, N-Tris(hydroxymethyl)acrylamide (ACRYLNTRIS-OH), glyceryl monomethacrylate (GLY-METH), poly(serine)methacrylate (SER-METH) or hydroxyethyl methacrylate (HEMA). In embodiments these base compounds can be cross-linked. Cross-linkers can be, for example, N,N′-(1,2-dihydroxyethylene) bisacrylamide, triglycerol diacrylate(glycerol 1,3-diglycerolate diacrylate TGDA), or tetraethylene glycol dimetharcrylate (TEGDMA). Crosslinkers can be interchanged in the different embodiments of the base matrix. Acrylate and methacrylate monomers may be synthesized as known in the art or obtained from a commercial vendor, such as Polysciences, Warrington, Pa. Inc., Sigma Aldrich, Inc., St.Louis, Mo. and Sartomer, Inc., Exton , Pa. Polypeptides may be synthesized as known in the art (or alternatively produced through molecular biological techniques) or obtained from a commercial vendor, such as American Peptide, Sunnyvale, CAGenScript Corporation, Piscataway, N.J. and Genway Biotech, Inc, San Diego, Calif. Spacers may be synthesized as known in the art or obtained from a commercial vendor, such as discrete polyethylene glycol (dPEG) spacers available from Quanta BioDesign, Ltd. Embodiments of the cell culture surface of the present invention are shown in Table 2.

TABLE 2

| BASE | | | |
|---|---|---|---|
| HYDROPHILIC | HYDROPHILIC | HYDROPHILIC | PEPTIDE- |
| MATRIX ID | MONOMER | CROSSLINKER | METHACRYLATE |
| ACRYLNTRIS-OH (1) | 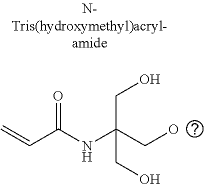 | 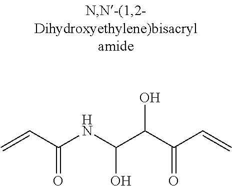 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |
| GLY-METH(2) | 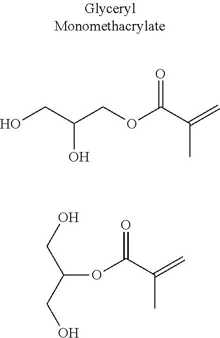 | 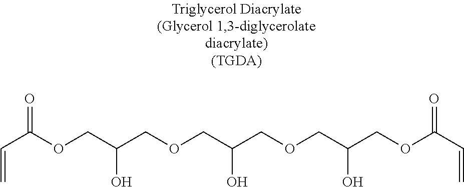 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |
| SER-METH(3) | 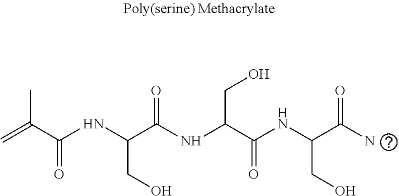 | 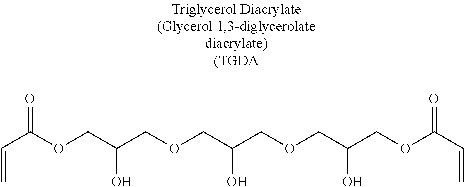 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |
| HEMA(4) | 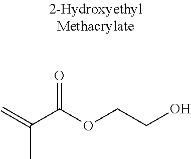 | 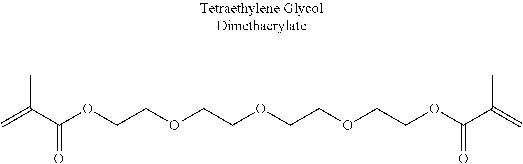 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |
| SORBITOL(5) | 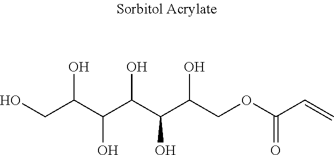 | 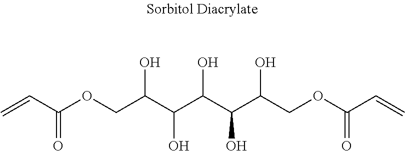 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |
| PENTAERYTHRITOL (6) | 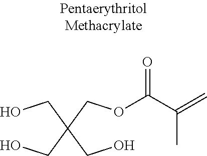 | 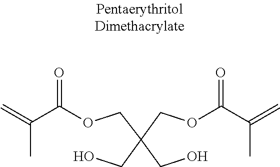 | MAA-PEG₄-Lys-Gly-Gly- Pro-Gln-Val-Thr-Arg-Gly- Asp-Val-Phe-Thr-Met- Pro-NH2 Ac-Lys(MAA)-Gly-Gly- Pro-Gln-Val-Thr-Arg-Gly- Asp-Val-Phe-Thr-Met- Pro-NH2 MAA-Lys-Gly-Gly-Pro- Gln-Val-Thr-Arg-Gly-Asp- Val-Phe-Thr-Met-Pro- NH2 |

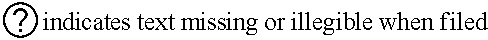

In embodiments, the base material may be dispensed onto a substrate, along with a cross-linker In embodiments, the base material may dispensed onto a substrate in lower alcohols such as ethanol. The base material may be polymerized, along with a cross-linker, by any polymerizing method. For example, the base polymeric material may be polymerized by exposure to UV, visible or thermal energy sources. For example, the base polymeric material may be polymerized using a 10s or 30s UV cure time. In embodiments, the cure is incomplete. That is, the time of exposure to a polymerizing energy source is insufficient to effect full polymerization of the base polymeric material, resulting in incomplete polymerization or a lower extent of reaction. The remaining polymerizable groups are therefore available for linking functionalized peptides such as, for example, methacrylate functionalized RGD containing adhesive peptides. For example, in embodiments, a methacrylate containing base material, in the presence of a cross-linker, may be incompletely polymerized, resulting in the presence of methacrylate moieties of these surfaces being available after incomplete polymerization for linking acrylate or methacrylate functionalized RGD containing adhesive peptides.

In embodiments, the substrate may be any material suitable for culturing cells, including a ceramic substance, a glass, a plastic, a polymer or co-polymer, any combinations thereof, or a coating of one material on another. The base material may be flat or shaped. Such base materials include glass materials such as soda-lime glass, borosilicate glass, Vycor® glass, quartz glass; silicon; plastics or polymers, including dendritic polymers, such as poly(vinyl chloride), poly(vinyl alcohol), poly(methyl methacrylate), poly(vinyl acetate-co-maleic anhydride), poly(dimethylsiloxane)monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like. As used herein, “cyclic olefin copolymer” means a polymer formed from more than one monomer species, where at least one of the monomer species is a cyclic olefin monomer and at least one other monomer species is not a cyclic olefin monomer species. In many embodiments, cyclic olefin copolymers are formed from ethylene and norbonene monomers. Cyclic olefin copolymer resins are commercially available with trade name of TOPAS® Florence, Ky., from Boedeker Plastics, and Inc Zeonor Corporation, Japan. In embodiments, the substrate may be treated to enhance retention of the polymer matrix. For example, the substrate may be treated with chemical or plasma treatments which provide negative charge, positive charge, create a more hydrophilic surface, or create functional chemical groups that enhance the adhesion of the polymer matrix to the substrate. For example, such treatments may include hydrophobic or hydrophilic interactions, steric interactions, affinities or Vander Waal forces.

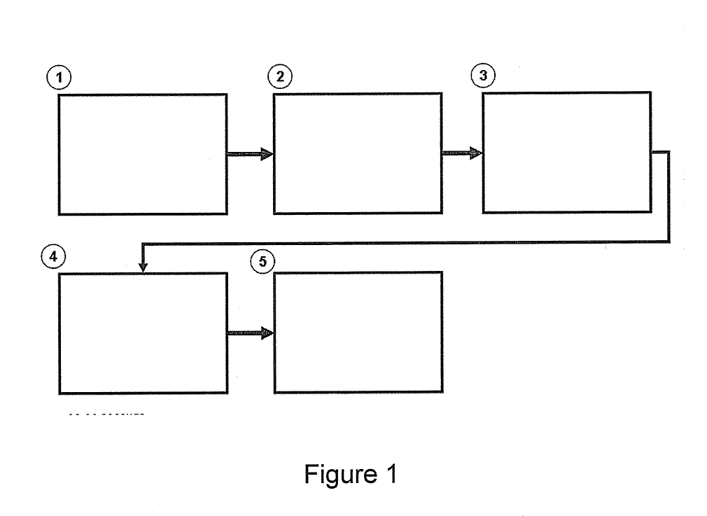

FIG. 1 is a flow chart showing an embodiment of a method of making cell culture surfaces. In embodiments, methods for providing cell binding peptides on the surface of a hydrophilic surface by photo-active chemical grafting are provided. These methods include steps of (1) providing a hydrophilic base material to a substrate surface; (2) curing or polymerizing the hydrophilic base material; (3) providing a functionalized peptide to the surface of cured hydrophilic base material; (4) curing or polymerizing the functionalized peptide to the hydrophilic base material; and optionally (5) washing to remove un-reacted monomers. In steps (1) and (3), the hydrophilic base material and the functionalized peptide may be provided to the surface of a substrate by any means know in the art including liquid dispensing, spin coating, spray coating, or other methods. In steps (2) and (4), the curing or polymerizing step may be accomplished by any means known in the art, and depending upon the nature of the polymerizing moiety, and may include the introduction of UV, visible or thermal energy to the surface. In step (5) washing may be accomplished by any means known in the art including liquid dispensing and incubating, with or without agitation, where the liquid may be water, a lower alcohol, a lower alcohol diluted in water, or other solvent.

In addition to the monomers that form the base material layer, a composition forming the layer may include one or more additional compounds such as surfactants, wetting agents, photoinitiators, thermal initiators, catalysts and activators. Any suitable polymerization initiator may be employed. One of skill in the art will readily be able to select a suitable initiator, e.g. a radical initiator or a cationic initiator, suitable for use with the monomers. In various embodiments, UV light is used to generate free radical monomers to initiate chain polymerization.

Any suitable initiator may be used. Examples of polymerization initiators include organic peroxides, azo compounds, quinones, nitroso compounds, acyl halides, hydrazones, mercapto compounds, pyrylium compounds, imidazoles, chlorotriazines, benzoin, benzoin alkyl ethers, diketones, phenones, or mixtures thereof. Examples of suitable commercially available, ultraviolet-activated and visible light-activated photoinitiators have tradenames such as IRGACURE 651, IRGACURE 184, IRGACURE 369, IRGACURE 819, DAROCUR 4265 and DAROCUR 1173 commercially available from Ciba Specialty Chemicals, Tarrytown, N.Y. and LUCIRIN TPO and LUCIRIN TPO-L commercially available from BASF (Charlotte, N.C.)

Additional initiators may include water soluble azo-initiators that can be used in thermal polymerization including, for example, (VA-044) 2,2′-Azobis[2-(2-imidazolin-2-yl)propane]dihydro chloride; (VA046B) 2,2′-Azobis[2-(2-imidazolin-2-yl)propane]disulfate dehydrate; (VA-50) 2,2′-Azobis(2-methylpropionamidine)dihydrochloride; (VA-057) 2,2′-Azobis[N-(2-carboxyethyl)-2-methylpropionamidine]hydrate; (VA-060) 2,2′-Azobis{2-[1-(2-hydroxyethyl)-2-imidazolin-2-yl]propane}dihydrochloride; (VA-061) 2,2′-Azobis[2-(2-imidazolin-2-yl)propane]; (VA-067) 2,2′-Azobis(1-imino-1-pyrrolidino-2-ethylpropane)dihydrochloride; (VA-080) 2,2′-Azobis{2-methyl-N-[1,1-bis(hydroxymethyl)-2-hydroxyethl]propionamide or (VA-086) 2,2′-Azobis[2-methyl-N-(2-hydroxyethyl)propionamide]. Oil soluble azo-initiators such as (V-70) 2,2′-Azobis(4-methoxy-2.4-dimethyl valeronitrile); (V-65) 2,2′-Azobis(2.4-dimethyl valeronitrile); (V-601) Dimethyl 2,2 ‘-azobis(2-methylpropionate); (V-59) 2,2’-Azobis(2-methylbutyronitrile; (V-40) 1,1′-Azobis(cyclohexane-1-carbonitrile); (VF-096) 2,2′-Azobis[N-(2-propenyl)-2-methylpropionamide]; (V-30) 1-[(1-cyano-1-methylethyl)azo]formamide; (VAm-110) 2,2′-Azobis(N-butyl-2-methylpropionamide) or (VAm-111) 2,2′-Azobis(N-cyclohexyl-2-methylpropionamide) may also be used in thermal polymerization. These initiators are available from for example, WAKO Chemicals, Richmond VA. In addition, macro-initiators, such as azo-initiators having a PEG backbone may be used in thermal polymerization.

A photosensitizer may also be included in a suitable initiator system. Representative photosensitizers have carbonyl groups or tertiary amino groups or mixtures thereof. Photosensitizers having a carbonyl groups include benzophenone, acetophenone, benzil, benzaldehyde, o-chlorobenzaldehyde, xanthone, thioxanthone, 9,10-anthraquinone, and other aromatic ketones. Photosensitizers having tertiary amines include methyldiethanolamine, ethyldiethanolamine, triethanolamine, phenylmethyl-ethano lamine, and dimethylaminoethylbenzo ate. Commercially available photosensitizers include QUANTICURE ITX, QUANTICURE QTX, QUANTICURE PTX, QUANTICURE EPD from Biddle Sawyer Corp, Crawley, England.

In general, the amount of photosensitizer or photoinitiator system may vary from about 0.01 to 10% by weight.

Examples of cationic initiators include salts of onium cations, such as arylsulfonium salts, as well as organometallic salts such as ion arene systems.

In various embodiments where the monomers are diluted in solvent before being deposited on the substrate surface, the solvent is removed prior to polymerizing. The solvent may be removed by any suitable mechanism or process. As described in copending U.S. application Ser. No. 12/362,782, it has been found that removal of substantially all of the solvent prior to curing, allows for better control of curing kinetics and the amount of monomer converted. When conversion rates of the monomers are increased, waste generation and cytotoxicity are reduced. Using these methods, the resulting base material layer forms a network, but not an interpenetrating network.

To form the synthetic base material, the monomers are polymerized. Whether polymerized in bulk phase (substantially solvent free) or solvent phase, the monomers are polymerized via an appropriate initiation mechanism. Many of such mechanisms are known in the art. For example, temperature may be increased to activate a thermal initiator, photoinitiators may be activated by exposure to appropriate wavelength of light, or the like. According to numerous embodiments, the monomer or monomer mixture is cured using UV light. The curing preferably occurs under inert gas protection, such as nitrogen protection, to prevent oxygen inhibition. Suitable UV light combined with gas protection may increase polymer conversion, insure coating integrity and reduce cytotoxicity.

In embodiments, the hydrophilic base material layer may be washed with solvent one or more times to remove impurities such as unreacted monomers or low molecular weight polymer species. In various embodiments, the layer is washed with ethanol or an ethanol-water solution, e.g. 70% ethanol, greater than 90% ethanol, greater than 95% ethanol or greater than about 99% ethanol. Washing with a 70% ethanol solvent may not only serve to remove impurities, which may be cytotoxic, but also can serve to sterilize the surface prior to incubation with cells.

In embodiments, the hydrophilic base material may be provided to a substrate surface and then semi-polymerized. That is, the polymerization process may be controlled or stopped before the base material is fully polymerized. Then a functionalized peptide may be provided to the surface of the semi-polymerized hydrophilic base material on the substrate surface. A second polymerization step may then be applied to polymerize the functionalized peptide to the semi-polymerized hydrophilic base material. The cell culture article may then be washed. The washing step may remove unpolymerized materials.

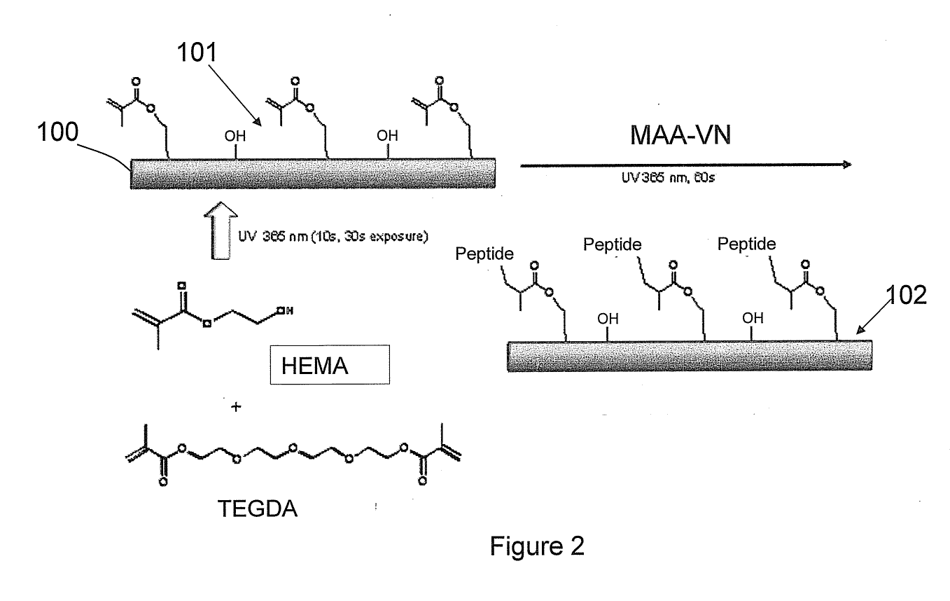

FIG. 2 is an illustration showing a method for making an embodiment of a cell culture surface. In FIG. 2, Tetraethylene glycol dimethacrylate and hydroxyl ethyl methacrylate (HEMA) are applied to a substrate and exposed to UV radiation at 365 nm for 10 seconds or 30 seconds to form a semi-polymerized base material or base layer. The functionalized peptide (Ac-Lys-(MAA)-Gly-Gly-Pro-Gln-Val-Thr-Arg-Gly-Asp-Val-Phe-Thr-Met-Pro-NH₂) Ac-Lys-(MAA)-SEQ ID NO:28)-NH₂ is then applied to the base layer and exposed to UV radiation at 365 nm for 60 seconds to form the HEMA base material with PEO₄ grafted functionalized peptide. Referring to Formula 1: Rₘ—Sₚ—Cₐₚ, Rₘ is methacrylic acid (MAA), Sₚ is present as Xaaₙ, where Xaa is Lys and n=1, methacrylic acid is attached to lysine through its amino acid side chain, and the cell adhesive peptide (Cₐₚ) is Seq ID NO: 28.

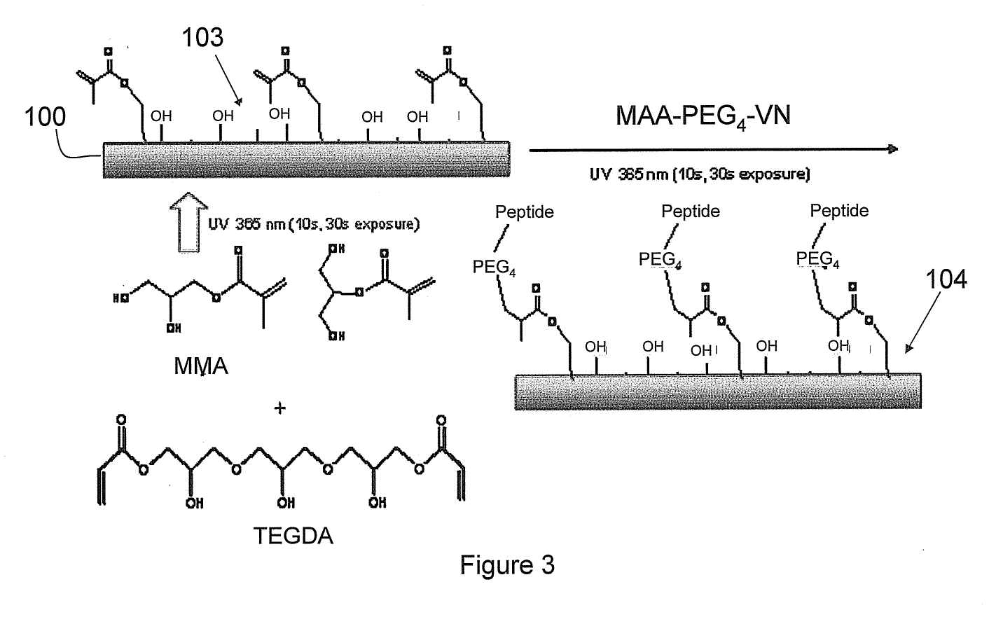

FIG. 3 is another illustration showing a method for making an embodiment of a cell culture surface. In FIG. 3, monomers triglycerol diacrylate (Glycerol 1,3-diglycerol diacrylate) and monomethacrylate isomers are deposited on a substrate and exposed to UV radiation at 365 nm for 10 seconds or 30 seconds to form a semi-polymerized base material or base layer. The functionalized peptide (MAA)-PEG₄-Lys-Gly-Gly-Pro-Gln-Val-Thr-Arg-Gly-Asp-Val-Phe-Thr-Met-Pro-NH₂) (MAA)-PEG₄-SEQ ID NO:27)-NH₂ is then applied to the base layer and exposed to UV radiation at 365 nm for 60 seconds to form a Glycerol Methacrylate base material with PEO₄ grafted functionalized peptide. Referring to Formula 1: Rₘ—Sₚ—Cₐₚ, Rₘ is methacrylic acid (MAA), spacer, Sₚ is present and is PEG₄, Xaa is absent, and the cell adhesive peptide is Seq ID NO: 27.

In embodiments, the cell culture surface may be formed on any surface suitable for cell culture. Examples of articles suitable for cell culture include single and multi-well plates, such as 6, 12, 96, 384, and 1536 well plates, jars, petri dishes, flasks, beakers, plates, roller bottles, slides, such as chambered and multi-chambered culture slides, tubes, cover slips, bags, membranes, hollow fibers, beads and micro-carriers, cups, spinner bottles, perfusion chambers, bioreactors, CellSTACK® (Corning, Incorporated) and fermenters.

Cells may be used for any suitable purpose, including (i) obtaining sufficient amounts of undifferentiated stem cells cultured on a synthetic surface in a chemically defined medium for use in investigational studies or for developing therapeutic uses, (ii) for investigational studies of the cells in culture, (iii) for developing therapeutic uses, (iv) for therapeutic purposes, (v) for studying gene expression, e.g. by creating cDNA libraries, and (vi) for studying drug and toxicity screening.

Cell culture articles prepared according to embodiments of the methods of the present invention can be effectively presented on the interface of a hydrophilic surface to facilitate growth and proliferation of any relevant cell type, including, for example, stem cells, adult stem cells, Embryonic Stem Cells (ESCs), human Embryonic Stem Cells (hESCs) or Inducible Pluripotent cells (IPCs). In embodiments, these cells in culture may be used in therapeutic applications. Because human embryonic stem cells (hESC) have the ability to grown continually in culture in an undifferentiated state, the hESC for use in this invention may be obtained from an established cell line. Examples of human embryonic stem cell lines that have been established include, but are not limited to, H1, H7, H9, H13 or H14 (available from WiCell established by the University of Wisconsin) (Thompson (1998) Science 282:1145); hESBGN-01, hESBGN-02, hESBGN-03 (BresaGen, Inc., Athens, Ga); HES-1, HES-2, HES-3, HES-4, HES-5, HES-6 (from ES Cell International, Inc., Singapore); HSF-1, HSF-6 (from University of California at San Francisco); I 3, I 3.2, I 3.3, I 4, I 6, I 6.2, J 3, J 3.2 (derived at the Technion-Israel Institute of Technology, Haifa, Israel); UCSF-1 and UCSF-2 (Genbacev et al., Fertil. Steril. 83(5):1517-29, 2005); lines HUES 1-17 (Cowan et al., NEJM 350(13):1353-56, 2004); and line ACT-14 (Klimanskaya et al., Lancet, 365(9471):1636-41, 2005). Embryonic stem cells used in the invention may also be obtained directly from primary embryonic tissue. Typically this is done using frozen in vitro fertilized eggs at the blastocyst stage, which would otherwise be discarded.

Other suitable stem cells include induced primate pluripotent (iPS) stem cells OPCs according to the invention may also be differentiated from induced primate pluripotent stem (iPS) cells. iPS cells refer to cells, obtained from a juvenile or adult mammal, such as a human, that are genetically modified, e.g., by transfection with one or more appropriate vectors, such that they are reprogrammed to attain the phenotype of a pluripotent stem cell such as an hESC. Phenotypic traits attained by these reprogrammed cells include morphology resembling stem cells isolated from a blastocyst as well as surface antigen expression, gene expression and telomerase activity resembling blastocyst derived embryonic stem cells. The iPS cells typically have the ability to differentiate into at least one cell type from each of the primary germ layers: ectoderm, endoderm and mesoderm and thus are suitable for differentiation into a variety of cell types. The iPS cells, like hESC, also form teratomas when injected into immuno-deficient mice, e.g., SCID mice. (Takahashi et al., (2007) Cell 131(5):861; Yu et al., (2007) Science 318:5858).

Embodiments of the present invention provide for efficient techniques for decorating surfaces with cell binding adhesive ligands such as peptides in a cost effective manner and facile manufacturing processes leading to overall significant reduction in manufacturing costs. In embodiments, the surfaces are useful surfaces for culturing cells, including human embryonic stem cells in the pluripotent (having more than one potential outcome) state using chemically defined media. The use of chemically defined media, in combination with synthetic surfaces in embodiments of the present invention is important because the use of serum introduces undefined factors into cell culture which may be detrimental to cultured cells intended for therapeutic uses.

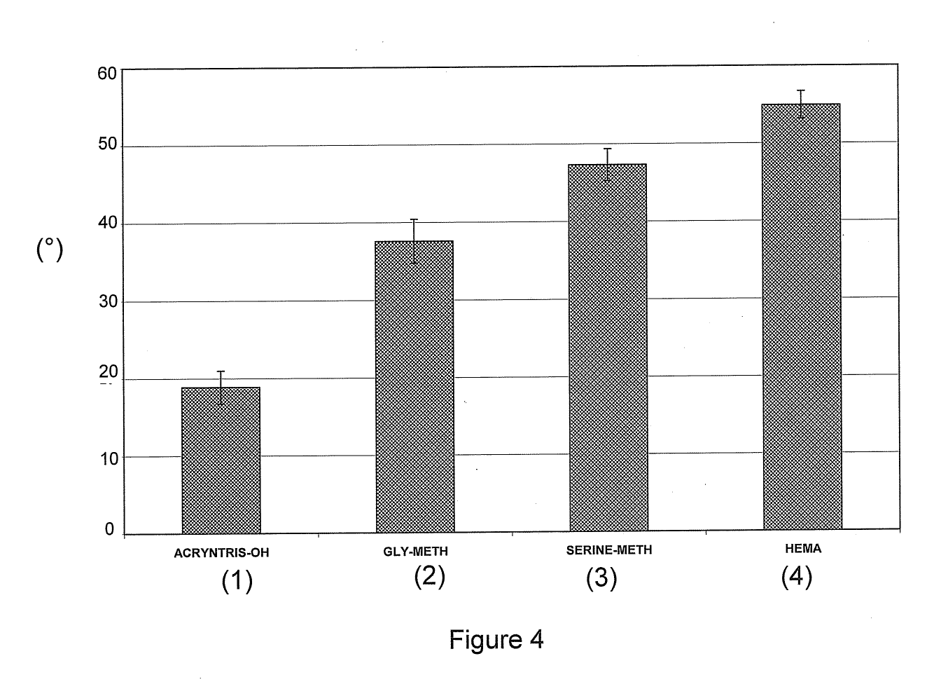

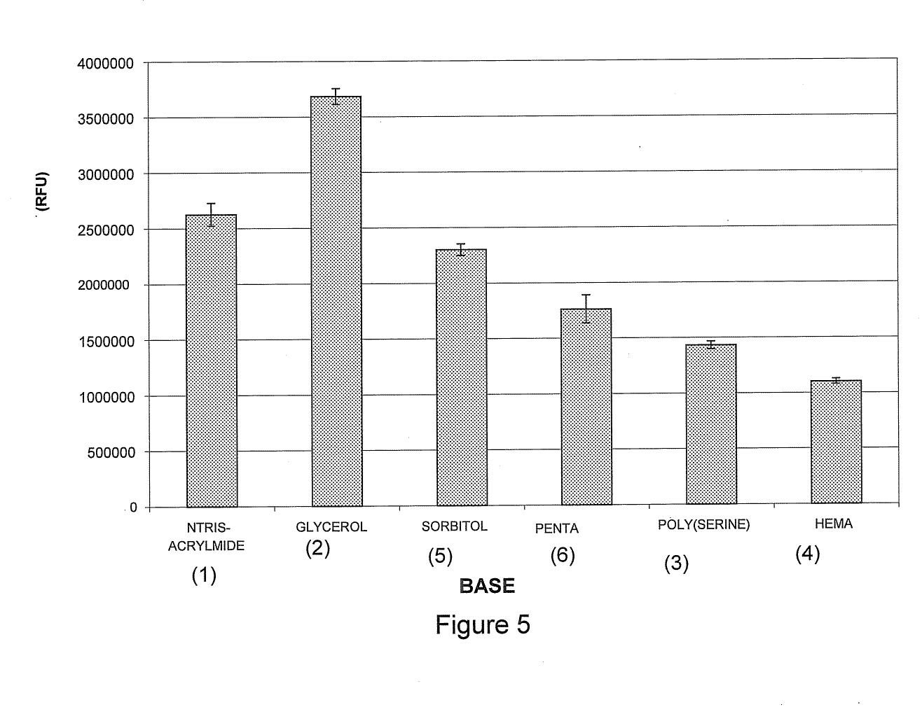

The surfaces created can be modeled based on their chemical structure to produce surfaces with high surface energy or low/receding contact angle that result in efficient dispersion of a photo-active PEO containing RGD adhesive peptide on the hydrophilic surfaces. FIG. 4 is a bar graph showing contact angles measured from the base materials (1-4) shown in Table 2. In embodiments, the surfaces have a water contact angle less than 60 degrees, between 15 and 60 degrees, between 20 and 60 degrees, greater than 15 degrees or greater than 20 degrees. FIG. 5 shows the fluorescence intensity of peptide (having PEO spacer). FIG. 5 shows fluorescence of MAA-PEO₄-Lys-Gly-Gly-Pro-Gln-Val-Thr-Arg-Gly-Asp-Val-Phe-Thr-Met-Pro-NH₂ (MAA-PEO₄-SEQ ID NO:27-NH₂) doped with 0.2% of rhodamine labeled version conjugated to six different base matrices, all of which are described in Table 2: N-Tris-Acrylamide (1) Glycerol Methacrylate (2), Sorbitol (3), Pentaerythritol (4), Poly-Serine (5) and HEMA (6). The more hydrophilic glycerol base matrix and NTRIS-Methacrylate surfaces had greater peptide grafting efficiency than the less hydrophilic HEMA and pentaerythritol methacrylate base matrix. Without being limited by theory, the PEO₄ spacer provided greater accessibility and therefore render improved peptide grafting efficiency over the peptide without spacer. It was observed that surfaces with lower contact angle provided for more efficient spreading of the functionalized peptide monomer on the base, and lower consumption of the photo-active peptide. This was even more pronounced when the lower contact angle base materials were combined with peptides contained a PEO₄ spacer as indicated by a MAA-PEG₄-Lys-Gly-Gly-Pro-Gln-Val-Thr-Arg-Gly-Asp-Val-Phe-Thr-Met-Pro-NH₂ (MAA-PEG₄-SEQ ID NO:27-NH₂) spiked with fluorescently labeled peptide. As used herein, contact angle refers to the initial contact angle of water on the substrate, without peptide.

In an aspect (1), a functionalized peptide comprising a composition of the formula Rₘ—Sₚ—Cₐₚ wherein R is a polymerization moiety selected from the group consisting of acrylate, methacrylate, acrylamide, methyacrylamide, maleimide fumarate and epoxide and combinations, and m is an integer greater than 1; and, wherein Sₚ is a spacer moiety wherein the spacer moiety comprises polyethylene oxide or polypropylene oxide having the formula O—CH₂CHR)ₘ₂ where R′ is H or CH₃ and m2 is an integer from 1 to 20, or Xaaₙ wherein Xaa is independently any amino acid and n is an integer from 0 to 3, or a combination; and wherein Cₐₚ is a peptide comprising a cell adhesive sequence is provided. In an aspect (2) the functionalized peptide of aspect 1 wherein Sₚ is a Lys or Arg amino acid is provided. In an aspect (3) the functionalized peptide of aspect for 2 wherein the cell adhesive peptide (Cₐₚ) comprises the sequence: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); KGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT(SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP(SEQ ID NO:28); GRGDSPK (SEQ ID NO:29); KGGAVTGRGDSPASS(SEQ ID NO:30); GGAVTGRGDSPASS (SEQ ID NO:31) or Yaa₁PQVTRGNVFTMP (SEQ ID NO:32) RGDYK(SEQ ID NO:33) is provided. In an aspect (4) the functionalized peptide of aspect 1 wherein the cell adhesive peptide (Cₐₚ) comprises KGGPQVTRGDVFTMP (SEQ ID NO:27) or GGPQVTRGDVFTMP(SEQ ID NO:28) is provided. In an aspect (5) the functionalized peptide of aspects 1-4 are provided wherein the polymerization moiety (Rₘ) comprises an acrylate or a methacrylate. In an aspect (6), the functionalized peptide of any one of aspects 1-5 is provided wherein Xaa comprises Lys and n=1.

In an additional aspect (7), a cell culture article comprising the functionalized peptide if claim 1 covalently linked to a hydrophilic polymeric base material, wherein the hydrophilic polymeric base material comprises N-Tris(hydroxymethyl)acrylamide (ACRYLNTRIS-OH) and N,N′-(1,2-dihydroxyethylene)bisacrylamide copolymer, glyceryl monomethacrylate and glycerol 1,3-diglycerolate diacrylate copolymer (GLY-METH) or poly(serine)methacrylate and glycerol 1,3-diglycerolate diacryate copolymer (SER-METH), hydroxyethylmethylacrylate (HEMA), or acrylamide (ACRYL) polymers and copolymers and optionally hydrophilic crosslinking materials such as, N,N′-(1,2-dihydroxyethylene)bisacrylamide, glycerol 1,3-diglycerolate diacrylate, or combinations thereof is provided. In an aspect (8), the cell culture article of aspect 7 is provided wherein the cell culture article has a contact angle of less than 50°. In an aspect, the cell culture article of aspect 7 is provided wherein the hydrophilic polymeric base material has a contact angle greater than 20°. In an aspect, the cell culture article of aspect 7 is provided wherein the hydrophilic polymeric base material has a contact angle between 20° and 60°. In an aspect, the cell culture article of aspect 7 is provided wherein the hydrophilic polymeric base material has a contact angle between 20° and 60°. In an aspect (9), the cell culture article of aspect 7 or 8 is provided wherein the cell adhesive peptide is selected from the group consisting of KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); CGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GRGDSPK (SEQ ID NO:29); KGGAVTGRGDSPASS (SEQ ID NO:30); GGAVTGRGDSPASS (SEQ ID NO:31); Yaa₁PQVTRGNVFTMP (SEQ ID); RGDYK(SEQ ID NO:33) or combinations. In an aspect (10), the cell culture article of any one of aspects 7-9 is provided wherein the spacer Sₘ comprises PEO₄. In an additional aspect (11), a method of making the cell culture article of claim 7 comprising the steps of: providing a hydrophilic base material to a substrate surface; semi-polymerizing the hydrophilic base material; providing a functionalized peptide to the surface of the semi-polymerized hydrophilic base material; polymerizing the functionalized peptide to the semi-polymerized hydrophilic base material; and, optionally washing is provided. In an aspect (12), the method of aspect 11 wherein the hydrophilic base material comprises N-Tris(hydroxymethyl)acrylamide (ACRYLNTRIS-OH) and N,N′-(1,2-dihydroxyethylene)bisacrylamide copolymer, glyceryl monomethacrylate and glycerol 1,3-diglycerolate diacrylate copolymer (GLY-METH) or poly(serine)methacrylate and glycerol 1,3-diglycerolate diacryate copolymer (SER-METH). In an aspect (13), the method of aspect 11 or 12 is provided wherein the semi-polymerized hydrophilic base material has a water contact angle of less than 50°. In an aspect (14), the method of any one of aspects 11-13 is provided, wherein Xaa comprises Lys and n=1. In an aspect (15), the method of any one of aspects 11-14 is provided wherein Rₘ comprises an acrylate or methacrylate. In an aspect (16), the method of any one of aspects 11-15 is provided, wherein Sₚ comprises PEO₄. In an aspect (17), the method of any one of aspects 11-16 is provided, wherein wherein Cₐₚ is a cell adhesive peptide selected from the group consisting of: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); CGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GRGDSPK (SEQ ID NO:29); KGGAVTGRGDSPASS (SEQ ID NO:30); GGAVTGRGDSPASS (SEQ ID NO:31); Yaa₁PQVTRGNVFTMP (SEQ ID NO:32); RGDYK(SEQ ID NO:33) and combinations.

In the following, non-limiting examples are presented, which describe various embodiments of the articles and methods discussed above.

### EXAMPLES

Abbreviations: (GDGMDMA)—Glycerol 1,3-Diglycero late, Dimethacrylate, (GMMA)-Glycerol monomethacrylate, TEGDMA—Tetraethyleneglycol dimethacrylate, HEMA—2-hydroxyethyl methacrylate, ACRYLNTRIS: (N-Tris(hydroxymethyl)acrylamide+N,N′-(1,2-dihydroxyethylene)bisacrylamide, also NTRIS-ACRYLAMIDE), SER-METH: (Poly(serine)4Methacrylate+Glycerol 1,3-Diglycerolate, GLY-METH: (Glycerol monomethacrylate+Glycerol 1,3-Diglycerolate Dimethacrylate, also shown as Glycerol in Table 2), (2-hydroxyethyl methacrylate+Tetraethyleneglycol dimethacrylate), EtOH—Ethanol, 1-819, Darocur 1173.

Materials: Photoinitiators Irgacure-819 (Phosphine oxide, phenyl bis(2,4,6-trimethyl benzoyl) and Darocur 1173 (2-hydroxy-2-methyl-1-phenyl-1-propanone) used in the free radical polymerization of the formulations were obtained from Ciba Specialty Chemicals (Newport Delaware) and used without any further purification. Hydrophilic crosslinkers, tetraethylene glycol dimethacrylate (86680), (454982) and glycerol 1,3-diglycerol diacrylate (475807) N,N′-(1,2-dihydroxyethylene)bisacrylamide (37474) were all purchased from Sigma-Aldrich in the purity as described in product specification sheet. Hydrophilic monomers 2-hydroxyethylmethacrylate, +99% (477028) and N-Tris(hydroxymethyl)acrylamide were purchased from Sigma-Aldrich while the other hydrophilic monomer used in the formulations, glycerol monomethacrylate isomers (04180) was purchased from Polysciences Incorporated without further purification. Poly(Serine)₄ Methacrylate used as a hydrophilic methacrylate functionalized polyamino acid along with adhesive peptides Ac-Lys(MAA)-Gly-Gly-Pro-Gln-Val-Thr-Arg-Gly-Asp-Val-Phe-Thr-Met-Pro-NH₂ (SEQ ID NO:27) and (MAA-PEO₄-VN): MAA-PEO₄-Lys-Gly-Gly-Pro-Gln-Val-Thr-Arg-Gly-Asp-Val-Phe-Thr-Met-Pro-NH₂ (SEQ ID NO:27) were synthesized by American Peptide, Sunnyvale, Calif. by the following processes.

General Process for the Synthesis of Functionalized Peptides:

Preparation of Ac-Lys(MAA)-Gly-Gly-Pro-Gln-Val-Thr-Arg-Gly-Asp-Val-Phe-Thr-Met-Pro-NH₂ (SEQ ID NO:27): The peptide was synthesized by American Peptide Sunnyvale, Calif. on 15 mmol Fmoc-Rink Amide resin via Fmoc chemistry. Protecting groups used for amino acids are: t-Butyl group for and Asp and Thr, Trt group for Gln, Pbf for Arg, Ivdde for Lys. Fmoc protected amino acids were purchased from EMD Biosciences. Reagents for coupling and cleavage were purchased from Aldrich. Solvents were purchased from Fisher Scientific. The peptide chain was assembled on resin by repetitive removal of the Fmoc protecting group and coupling of protected amino acid. DIC and HOBt were used as coupling reagents and NMM was used as base. 20% piperidine in DMF was used as de-Fmoc-reagent. Methacrylic acid (MAA) was coupled on the side chain of Lysine after Ivdde was removed by 2% Hydrazine in DMF. After the last coupling, resin was treated with TFA/TIS/H2O (95:3:2, v/v/v) for cleavage and removal of the side chain protecting groups. Crude peptide was precipitated from cold ether and collected by filtration. Yield 33.0 gram (Synthesis yield 194.2%).17 g crude peptide was purified by reverse-phase HPLC; collected fractions with purity over 90% were pooled and lyophilized. Yield final product 9.25 g (purification yield 54.4%).

Preparation of (MAA-PEO₄-VN): MAA-PEO₄-Lys-Gly-Gly-Pro-Gln-Val-Thr-Arg-Gly-Asp-Val-Phe-Thr-Met-Pro-NH₂ (SEQ ID NO:27): The peptide was synthesized by American Peptide Sunnyvale, Calif. on 1 mmol Fmoc-Rink Amide resin via Fmoc chemistry. Protecting groups used for amino acids are: t-Butyl group for and Asp and Thr, Trt group for Gln, Pbf for Arg, Boc for Lys. Fmoc protected amino acids were purchased from EMD Biosciences; Fmoc-PEG4-OH was purchased from Quanta Biodesign. Reagents for coupling and cleavage were purchased from Aldrich. Solvents were purchased from Fisher Scientific. The peptide chain was assembled on resin by repetitive removal of the Fmoc protecting group and coupling of protected amino acid. HBTU and HOBt were used as coupling reagents and NMM was used as base. 20% piperidine in DMF was used as de-Fmoc-reagent. Methacrylic acid (MAA) was coupled to the amino group of PEG4 after removal of the Fmoc protecting group. After the last coupling, resin was treated with TFA/TIS/H2O (95:3:2, v/v/v) for cleavage and removal of the side chain protecting groups. Crude peptide was precipitated from cold ether and collected by filtration. Yield 4.0 gram (Synthesis yield 210.1%). Crude peptide was purified by reverse-phase HPLC; collected fractions with purity over 90% were pooled and lyophilized. Yield final product 1.035 g (purification yield 25.9%).

The products were provided by American Peptide in ≧90% purity and were used without further purification. Ethanol was used as non-reactive diluent in the process and was purchased from Sigma-Aldrich.

General Procedure for the Preparation of Cell Culture Surfaces:

Into a 20 ml scintillation vial 4.37 mg (0.23 mM) of MAA-PEG₄-Lys-Gly-Gly-Pro-Gln-Val-Thr-Arg-Gly-Asp-Val-Phe-Thr-Met-Pro-NH₂ (MAA-PEG4-SEQ ID NO:27-NH₂) was added to 10 ml of ethanol. The other grafting peptide VN-MAA: Lys(MAA)-Gly-Gly-Pro-Gln-Val-Thr-Arg-Gly-Asp-Val-Phe-Thr-Met-Pro-NH₂ (Lys-Maa-SEQ ID NO:27-NH₂) in 8.74 mg (0.5 mM) was also prepared in similar fashion.

Into a separate 10 ml scintillation vial quantities of 400 μL of 2-hydroxyethyl methacrylate was added, subsequently ethanol was added along with 40 μL of tetra(ethylene glycol) dimethacrylate, 15 μL of Darocur 1173 (10% in ethanol), 50 μL Irgacure 819 (1% in ethanol) and 9.5 ml of ethanol. This recipe amounts to a 5% formulation in ethanol. For other hydrophilic libraries involving GLY-METH, SER-METH and ACRYLNTRIS-OH were all prepared in 5% formulations in ethanol. These stock solutions were used for coating in 6-well plates (6 wp).

General Procedure for Coating of Formulations in 6 wp:

Six-well plates (6 wp) were removed from packaging and placed in large nitrogen purge box which was continuously being purged with nitrogen gas. Into 6-wp, 26 μl of (5%) of the hydrophilic formulations listed in table 2 were placed into respective wells and the plates were placed in a vacuum oven for 5 min and then allowed to cure for 10 or 30 seconds. The surfaces were slightly tacky, indicating the presence of left over methacrylate groups. A semi-automated pipettor was used to dispense, onto tacky surface 200 μL or 500 μL of stock solution of MAA-PEG₄-Lys-Gly-Gly-Pro-Gln-Val-Thr-Arg-Gly-Asp-Val-Phe-Thr-Met-Pro-NH₂ (MAA-PEG₄-SEQ ID NO:27-NH₂), and Lys(MAA)-Gly-Gly-Pro-Gln-Val-Thr-Arg-Gly-Asp-Val-Phe-Thr-Met-Pro-NH₂ (Lys-MAA-SEQ ID NO:27-NH₂) into respective 6-wp per plate. The solution was allowed to spread for 15 minutes, then cured for 1 minute, then washed for 1 h in ethanol followed by 1 h in PBS buffer, then rinsed for 1 minute with DI water.

To prepare coatings for contact angle measurements, the base hydrophilic matrix only was applied to a 6wp substrate and cured for 10 or 30 seconds. Contact angle measurements were made on the base substrate. To coat samples to measure peptide grafting efficiency via fluorescence spectroscopy, 10 μL of Rhodamine-labeled peptide was added to 1 ml of peptide-methacrylate and coated on their respective hydrophilic base matrix. Surfaces were then cured for 60 seconds and surfaces were scanned before and after washing.

Measurement of Contact Angle: The water contact angle measurements were obtained on Rame-Hart goniometer (Rame-Hart Instrument Company, 95 Allen Street, Netcong, N.J., 07857-0400) using dI water and measured within one minute after the water was placed on the surface.

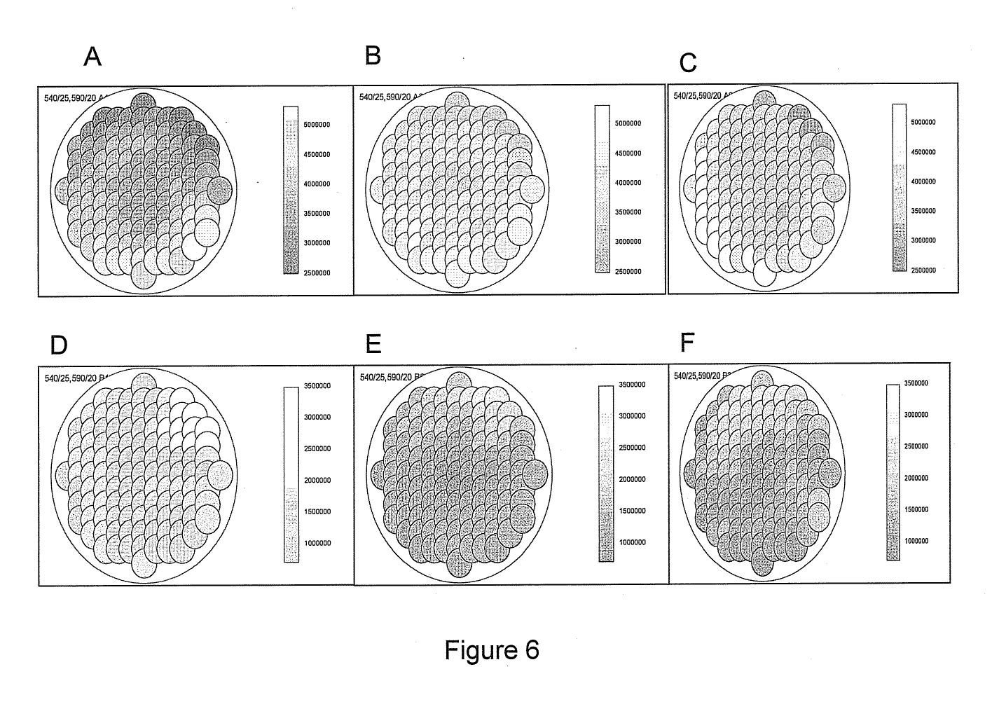

FIG. 6 illustrates fluorescence measurements taken from Rhodamine-labeled functionalized peptide (MAA-PEG₄-SEQ ID NO:27-NH₂) on a GLY-METH base material (FIGS. 6A-C) and on a HEMA surface (FIGS. 6D-F). The peptide grafting efficiency was 3× higher for the GLY-METH base material, which is more hydrophilic, compared to the HEMA base material.

Procedure for UV Curing Coatings

After the solvent was removed, the coatings were cured with 10˜50 mW/cm² pulsed (100 Hz) UV light (using a Xenon RC-801 system) for 1 min in N₂ purged box (with fused silica window). The distance between UV lamp and coating surface was 5±0.5 inch. Plates were cured for 60 seconds. Note that LED lighting can also be used. The cure chamber was constantly being purged with nitrogen which was necessary in order to create an inert environment (for the coatings) during curing.

Procedure for Washing Grafted Coatings

The Grafted surfaces (base materials with functionalized peptides applied) were washed with ethanol for 30-60 minutes followed by washing with PBS buffer for 30 minutes. The plates were dried overnight and submitted for cell testing.

Procedure for Culturing Human Embryonic Stem Cells:

H7 hES cells were provided as part of collaboration agreement with Geron Corporation (Menlo Park, Calif.) and cultured according to their protocols. Briefly, cells were cultured on MG-coated TCT flasks in chemically defined medium (X-Vivo-10, 80 ng/ml hbFGF, 5ng/ml hTGF-β1). Cells were passaged every 4-5 days at the seeding density of 10×10⁶ cells/T75 flask using Geron's sub-cultivation procedure. For the experiments, cells were seeded in 6-well plates at the density of 1×10⁶/well in chemically defined media. Cell morphology was observed daily. Cells were stained with crystal violet on day 4 for visual assessment of cell number, colony morphology, and distribution.

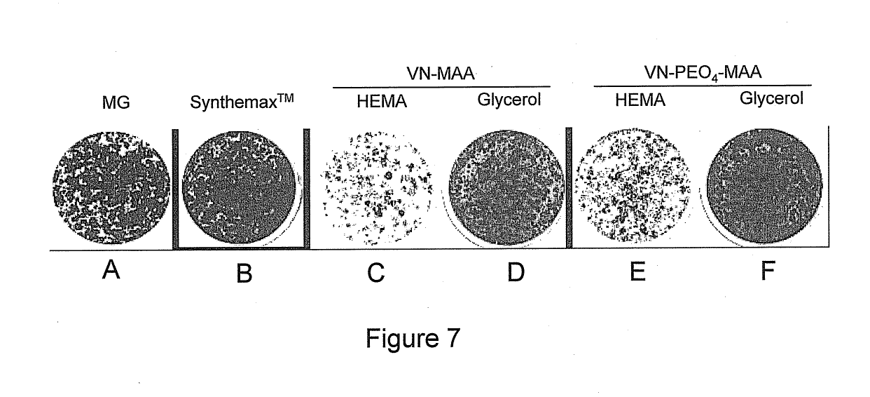

FIG. 7 shows photomicrographs of H7 human embryonic stem cells cultured on control surfaces Matrigel™ available from BD, Franklin Lakes N.J., (FIG. 7A), Synthemax™ available from Corning Incorporated, Corning, N.Y. (FIG. 7B), VN-MAA functionalized peptide on a HEMA base (FIG. 7C), VN-MAA functionalized peptide on a glycerol methacrylate (GLY METH) base (FIG. 7D), VN-PEO4-MAA on a HEMA base (FIG. 7E) and VN-PEO₄-MAA on a glycerol methacrylate (GLY METH) base (FIG. 7F). Functionalized peptide grafted to GLY-METH base material showed human stem cell growth and morphology similar to control after four days in culture, with both the (Lys-MAA-SEQ ID NO:27-NH₂) and the (MAA-PEG₄-SEQ ID NO:27-NH₂) functionalized peptides, compared to the less hydrophilic HEMA surface. Further, for both HEMA and GLYCEROL base matrix MAA-PEO₄-Lys-Gly-Gly-Pro-Gln-Val-Thr-Arg-Gly-Asp-Val-Phe-Thr-Met-Pro-NH₂ (MAA-PEG₄-SEQ ID NO:27-NH₂) or (VN-PEO₄-MAA) on HEMA or Glycerol (GLYC-METH) support better cell attachment and morphology when compared the respective VN-MAA.

The base matrix surfaces with lower contact angle (highly hydrophilic) for e.g. GLYC-METH (37.4°) resulted in higher density of MAA-PEG₄-Lys-Gly-Gly-Pro-Gln-Val-Thr-Arg-Gly-Asp-Val-Phe-Thr-Met-Pro-NH₂ (MAA-PEG₄-SEQ ID NO:27-NH₂) grafting than its HEMA counterpart with contact angle of (54.9°) and is considered a preferred embodiment for cell culturing of H7 hESCs. However, different cells may survive in cell culture on base materials having higher or lower contact angles, depending upon the cell culture preferences of that particular cell type. Moreover, a functionalized peptide, or peptide methacrylate with PEO₄ spacer length resulted in higher grafting efficiency than one without a PEO₄ spacer. Subsequently, the surfaces with more efficient peptide grafting and therefore higher peptide density were able to show significant improvement in cell adhesion and spreading of H7 Human Embryonic Stem Cells (hESCs) over 4 days of culturing. Without being limited by theory, it may be that a hydrophilic base surface and a functionalized peptide with PEO₄ spacer act synergistically to drive peptide grafting efficiency, and therefore, elicit higher cell response. Mass transport in and on the interface of the matrix may allow for efficient spreading during grafting of methacrylate functionalized peptides while the inclusion of a PEO₄ spacer on peptide may allow for providing ligand accessibility and proper orientation for putative cell attachment and spreading of human embryonic stem cells (hESCs). Increasing the hydrophilic nature of the base material may be responsible for several occurrences: 1) mass transport of methacrylate functionalized peptides are improved over the surface, 2) un-reacted methacrylates from deliberate under-curing of the base matrix are rendered more mobile on the surface interface and therefore have greater accessibility for connecting with methacrylates; and, 3) less volume of solution may be required to facilitate grafting due to hydrophilic tunability of these surfaces. Additional studies (not presented) conducted using different chemistries to impart hydrophilicity show a continued trend of increase grafting efficiency with decreasing contact angle.

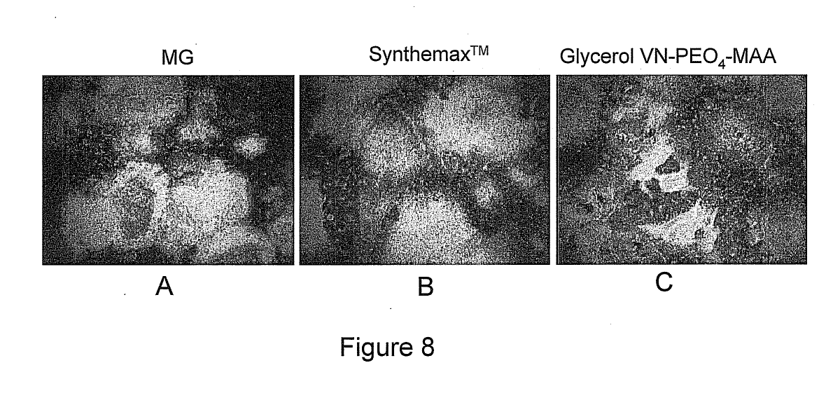

FIG. 8 shows photomicrographs of H7 human embryonic stem cells grown on control surfaces MG (Matrigel™), (FIG. 8A), Synthemax™ (FIG. 8B), and the glycerol-methacrylate VN-PEO4-MAA (GLY-METH) for four days. FIG. 8 illustrates that the morphology of H7 hESC cells cultured on embodiments of the cell culture surface of the present invention is comparable to Matrigel™ and Synthemax™.

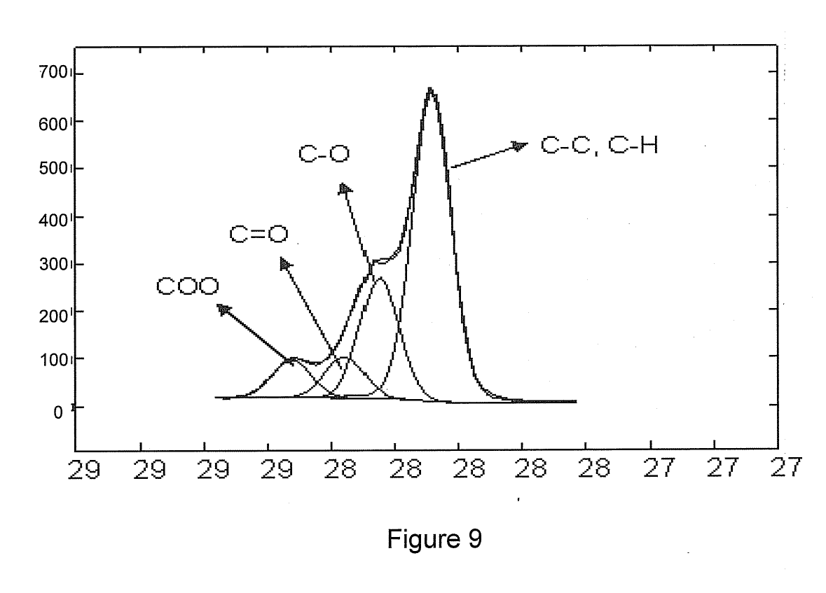

FIG. 9 shows XPS data showing binding energy of detected oxygen in HEMA surfaces. The atomic composition of the top 2-6 nm of the surfaces is shown in Tables 3 and 4. These data were collected based on only 2 different scanned areas, and using a lower resolution pass energy of ˜45 eV because there was a need to collect the data quickly in order to minimize the chance of beam or vacuum damage. The results of the XPS analysis for the HEMA surface are shown in Table 3.

TABLE 3

| | C1s | N1s | O1s | Na1s |
|---|---|---|---|---|
| Measurement Area 1 | 79.45 | 0.35 | 19.92 | 0.28 |
| Measurement Area 2 | 79.9 | 0.38 | 79.37 | 0.36 |
| Mean | 79.67 | 0.36 | 19.64 | 0.32 |
| St. Dev. | 0.31 | 0.02 | 0.39 | 0.06 |

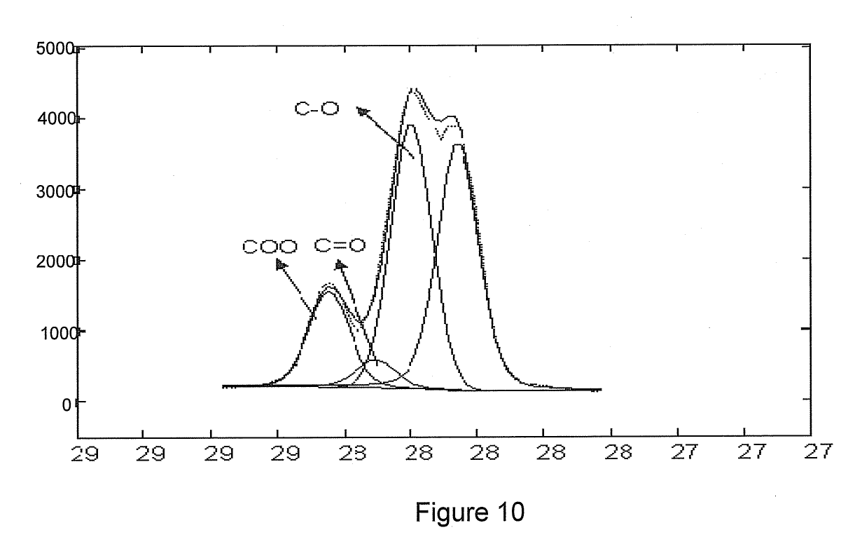

FIG. 10 shows XPS data showing binding energy of detected oxygen in GLY-METH surfaces. The results of XPS analysis are shown in Table 4:

TABLE 4

| | C1s | N1s | O1s |
|---|---|---|---|
| Measurement Area 1 | 63.62 | 0.11 | 36.27 |
| Measurement Area 2 | 65.86 | 0.03 | 34.1 |
| St Dev. | 1.59 | 0.06 | 1.53 |
| Mean | 64.74 | 0.07 | 35.19 |

XPS showed that the surface with a higher contact angle (acrylated glycol) displayed higher oxygen content. The ratio of oxygen in the GLYC-to-HEMA=35.2/19.6=1.78. In particular, the GLYC-METH surface showed a higher C—O peak than the HEMA surface. This is consistent with the presence of a greater number of OH groups on the surface of the GLY-METH surface. XPS also showed that these two surfaces contain different ratios of oxygen-containing functionalities. The high oxygen content of the glycerol surfaces is shown by the increased oxygen containing groups on the surface that is responsible for driving the higher surface energy (low/receding) contact angle that facilitates spreading of methacrylate functionalized peptide for grafting.

Thus, embodiments of FUNCTIONALIZED CELL BINDING PEPTIDES AND CELL CULTURE ARTICLES are disclosed. One skilled in the art will appreciate that the arrays, compositions, kits articles and methods described herein can be practiced with embodiments other than those disclosed. The disclosed embodiments are presented for purposes of illustration and not limitation.

What is claimed is:

1. A functionalized peptide comprising:
a composition of the formula Rₘ—Sₚ—Cₐₚ
wherein R is a polymerization moiety selected from the group consisting of acrylate, methacrylate, acrylamide, methyacrylamide, maleimide, fumarate and epoxide and combinations, and m is an integer equal to or greater than 1; and,
wherein Sₚ is a spacer moiety wherein the spacer moiety comprises polyethylene oxide or polypropylene oxide having the formula (O—CH₂CHR′)ₘ₂ where R′ is H or CH₃ and m2 is an integer from 1 to 20, or Xaaₙ wherein Xaa is independently any amino acid and n is an integer from 0 to 20, or a combination; and
wherein Cₐₚ is a peptide comprising a cell adhesive sequence.

2. The functionalized peptide of claim 1 wherein Sₚ comprises a Lys or Arg amino acid.

3. The functionalized peptide of claim 1 wherein the cell adhesive peptide (Cₐₚ) comprises the sequence: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); KGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT(SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP(SEQ ID NO:28); GRGDSPK (SEQ ID NO:29); KGGAVTGRGDSPASS(SEQ ID NO:30); GGAVTGRGDSPASS (SEQ ID NO:31) or Yaa₁PQVTRGNVFTMP (SEQ ID NO:32) RGDYK(SEQ ID NO:33).

4. The functionalized peptide of claim 1 wherein the cell adhesive peptide (Cₐₚ) comprises KGGPQVTRGDVFTMP (SEQ ID NO:27) or GGPQVTRGDVFTMP(SEQ ID NO:28).

5. The functionalized peptide of claim 1 wherein the polymerization moiety (Rₘ) comprises an acrylate or a methacrylate.

6. The functionalized peptide of claim 1 wherein Xaa comprises Lys and n is greater than or equal to 1.

7. A cell culture article comprising the functionalized peptide if claim 1 covalently linked to a hydrophilic polymeric base material, wherein the hydrophilic polymeric base material comprises a non-ionic hydrophilic polymer made from an acrylate, methacrylate, acrylamide, methyacrylamide, maleimide, fumarate or and epoxide and combinations thereof.

8. The cell culture article of claim 7 wherein the hydrophilic polymeric base material comprises non-ionic hydrophilic polymer made from (1) N-Tris(hydroxymethyl)acrylamide (ACRYLNTRIS-OH); (2) glyceryl monomethacrylate; (3) sorbitol; (4) pentaerythritol; (5) poly(serine)methacrylate (SER-METH); or (6) hydroxyethylmethylacrylate (HEMA) polymers and copolymers.

9. The cell culture article of claim 7 wherein the hydrophilic polymeric base material comprises non-ionic hydrophilic polymer made from (i) N-Tris(hydroxymethyl)acrylamide (ACRYLNTRIS-OH) and N,N′-(1,2-dihydroxyethylene)bisacrylamide copolymer; (ii) glyceryl monomethacrylate and glycerol 1,3-diglycerolate diacrylate copolymer (GLY-METH); (iii) poly(serine)methacrylate and glycerol 1,3-diglycerolate diacryate copolymer; (iv) hydroxyethylmethylacrylate (HEMA) polymers and copolymers, or (v) combinations thereof.

10. The cell culture article of claim 8 wherein the hydrophilic polymeric base material has a contact angle of less than 60°.

11. The cell culture article of claim 7 wherein the cell adhesive peptide is selected from the group consisting of KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); CGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GRGDSPK (SEQ ID NO:29); KGGAVTGRGDSPASS (SEQ ID NO:30); GGAVTGRGDSPASS (SEQ ID NO:31); Yaa₁PQVTRGNVFTMP (SEQ ID NO:32) RGDYK(SEQ ID NO:33); or combinations.

12. The cell culture article of claim 7 wherein the spacer Sₘ comprises PEO₄.

13. A method of making the cell culture article of claim 7 comprising the steps of:
providing a hydrophilic base material to a substrate surface;
semi-polymerizing the hydrophilic base material;
providing a functionalized peptide to the surface of the semi-polymerized hydrophilic base material;
polymerizing the functionalized peptide to the semi-polymerized hydrophilic base material; and, optionally washing.

14. The method of claim 13 wherein the hydrophilic base material comprises a non-ionic hydrophilic polymer made from an acrylate, methacrylate, acrylamide, methyacrylamide, maleimide, fumarate or and epoxide and combinations thereof.

15. The method of claim 13 wherein the hydrophilic base material comprises (1) N-Tris(hydroxymethyl)acrylamide (ACRYLNTRIS-OH); (2) glyceryl monomethacrylate; (3) sorbitol; (4) pentaerythritol; (5) poly(serine)methacrylate (SER-METH); or (6) hydroxyethylmethylacrylate (HEMA) polymers and copolymers.

16. The method of claim 13 wherein the semi-polymerized hydrophilic base material has a water contact angle of less than 60°.

17. The method of claim 13 wherein Xaa comprises Lys and n is greater or equal to 1.

18. The method of claim 13 wherein Rₘ comprises an acrylate or methacrylate.

19. The method of claim 13 wherein Sₚ comprises PEO₄.

20. The method of claim 11 wherein Cₐₚ is a cell adhesive peptide selected from the group consisting of: KGGGQKCIVQTTSWSQCSKS (SEQ ID NO:1); GGGQKCIVQTTSWSQCSKS(SEQ ID NO:2); KYGLALERKDHSG (SEQ ID NO:3); YGLALERKDHSG (SEQ ID NO:4); KGGSINNNRWHSIYITRFGNMGS (SEQ ID NO:5); GGSINNNRWHSIYITRFGNMGS (SEQ ID NO:6); KGGTWYKIAFQRNRK (SEQ ID NO:7); GGTWYKIAFQRNRK (SEQ ID NO:8); KGGTSIKIRGTYSER (SEQ ID NO:9); GGTSIKIRGTYSER (SEQ ID NO:10); KYGTDIRVTLNRLNTF (SEQ ID NO:11); YGTDIRVTLNRLNTF (SEQ ID NO:12); KYGSETTVKYIFRLHE (SEQ ID NO:13); YGSETTVKYIFRLHE (SEQ ID NO:14); KYGKAFDITYVRLKF (SEQ ID NO:15); YGKAFDITYVRLKF (SEQ ID NO:16); KYGAASIKVAVSADR (SEQ ID NO:17); YGAASIKVAVSADR (SEQ ID NO:18); CGGNGEPRGDTYRAY (SEQ ID NO:19); GNGEPRGDTYRAY (SEQ ID NO:20); CGGNGEPRGDTRAY (SEQ ID NO:21); GGNGEPRGDTRAY (SEQ ID NO:22); KYGRKRLQVQLSIRT (SEQ ID NO:23); YGRKRLQVQLSIRT (SEQ ID NO:24); KGGRNIAEIIKDI (SEQ ID NO:25); GGRNIAEIIKDI (SEQ ID NO:26); KGGPQVTRGDVFTMP (SEQ ID NO:27); GGPQVTRGDVFTMP (SEQ ID NO:28); GRGDSPK (SEQ ID NO:29); KGGAVTGRGDSPASS (SEQ ID NO:30); GGAVTGRGDSPASS (SEQ ID NO:31); Yaa₁PQVTRGNVFTMP (SEQ ID NO:32) RGDYK(SEQ ID NO:33); and combinations.


SEQUENCE LISTING (33 sequences)

SEQ ID NO: 1 - protein, 20 aa, Artificial Sequence
  Synthetic Peptide
KGGGQKCIVQTTSWSQCSKS

SEQ ID NO: 2 - protein, 19 aa, Artificial Sequence
  Synthetic Peptide
GGGQKCIVQTTSWSQCSKS

SEQ ID NO: 3 - protein, 13 aa, Artificial Sequence
  Synthetic Peptide
KYGLALERKDHSG

SEQ ID NO: 4 - protein, 12 aa, Artificial Sequence
  Synthetic Peptide
YGLALERKDHSG

SEQ ID NO: 5 - protein, 23 aa, Artificial Sequence
  Synthetic Peptide
KGGSINNNRWHSIYITRFGNMGS

SEQ ID NO: 6 - protein, 22 aa, Artificial Sequence
  Synthetic Peptide
GGSINNNRWHSIYITRFGNMGS

SEQ ID NO: 7 - protein, 15 aa, Artificial Sequence
  Synthetic Peptide
KGGTWYKIAFQRNRK

SEQ ID NO: 8 - protein, 14 aa, Artificial Sequence
  Synthetic Peptide
GGTWYKIAFQRNRK

SEQ ID NO: 9 - protein, 15 aa, Artificial Sequence
  Synthetic Peptide
KGGTSIKIRGTYSER

SEQ ID NO: 10 - protein, 14 aa, Artificial Sequence
  Synthetic Peptide
GGTSIKIRGTYSER

SEQ ID NO: 11 - protein, 16 aa, Artificial Sequence
  Synthetic Peptide
KYGTDIRVTLNRLNTF

SEQ ID NO: 12 - protein, 15 aa, Artificial Sequence
  Synthetic Peptide
YGTDIRVTLNRLNTF

SEQ ID NO: 13 - protein, 16 aa, Artificial Sequence
  Synthetic Peptide
KYGSETTVKYIFRLHE

SEQ ID NO: 14 - protein, 15 aa, Artificial Sequence
  Synthetic Peptide
YGSETTVKYIFRLHE

SEQ ID NO: 15 - protein, 15 aa, Artificial Sequence
  Synthetic Peptide
KYGKAFDITYVRLKF

SEQ ID NO: 16 - protein, 14 aa, Artificial Sequence
  Synthetic Peptide
YGKAFDITYVRLKF

SEQ ID NO: 17 - protein, 15 aa, Artificial Sequence
  Synthetic Peptide
KYGAASIKVAVSADR

SEQ ID NO: 18 - protein, 14 aa, Artificial Sequence
  Synthetic Peptide
YGAASIKVAVSADR

SEQ ID NO: 19 - protein, 15 aa, Artificial Sequence
  Synthetic Peptide
CGGNGEPRGDTYRAY

SEQ ID NO: 20 - protein, 14 aa, Artificial Sequence
  Synthetic Peptide
GGNGEPRGDTYRAY

SEQ ID NO: 21 - protein, 14 aa, Artificial Sequence
  Synthetic Peptide
CGGNGEPRGDTRAY

SEQ ID NO: 22 - protein, 13 aa, Artificial Sequence
  Synthetic Peptide
GGNGEPRGDTRAY

SEQ ID NO: 23 - protein, 15 aa, Artificial Sequence
  Synthetic Peptide
KYGRKRLQVQLSIRT

SEQ ID NO: 24 - protein, 14 aa, Artificial Sequence
  Synthetic Peptide
YGRKRLQVQLSIRT

SEQ ID NO: 25 - protein, 13 aa, Artificial Sequence
  Synthetic Peptide
KGGRNIAEIIKDI

SEQ ID NO: 26 - protein, 12 aa, Artificial Sequence
  Synthetic Peptide
GGRNIAEIIKDI

SEQ ID NO: 27 - protein, 15 aa, Artificial Sequence
  Synthetic Peptide
KGGPQVTRGDVFTMP

SEQ ID NO: 28 - protein, 14 aa, Artificial Sequence
  Synthetic Peptide
GGPQVTRGDVFTMP

SEQ ID NO: 29 - protein, 7 aa, Artificial Sequence
  Synthetic Peptide
GRGDSPK

SEQ ID NO: 30 - protein, 15 aa, Artificial Sequence
  Synthetic Peptide
KGGAVTGRGDSPASS

SEQ ID NO: 31 - protein, 14 aa, Artificial Sequence
  Synthetic Peptide
GGAVTGRGDSPASS

SEQ ID NO: 32 - protein, 16 aa, Artificial Sequence
  Synthetic Peptide
YAALPQVTRGNVFTMP

SEQ ID NO: 33 - protein, 5 aa, Artificial Sequence
  Synthetic Peptide
RGDYK